(19) United States
(12) Patent Application Publication
(10) Pub. No.: US 2010/0041662 A1
(43) Pub. Date: Feb. 18, 2010

(54) Heterocyclic compounds as antiinflammatory agents

(76) Inventors: Sandrine Ferrand, Huningue (FR); Fraser Glickman, Basel (CH); Catherine Leblanc, West Sussex (GB); Cathy Ritchie, West Sussex (GB); Duncan Shaw, West Sussex (GB); Nikolaus Johannes Stiefl, Lorrach (DE); Pascal Furet, Thann (FR); Patricia Imbach, Kaiseraugst (CH); Frédéric Stauffer, Hesingue (FR); Hans-Georg Capraro, Rheinfelden (CH); François Gessier, Altkirch (FR); Christoph Gaul, Aesch (CH); Pameia A. Albaugh, Carlsbad, CA (US); Greg Chopiuk, San Diego, CA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/311,393

(22) PCT Filed: Oct. 29, 2007
(86) PCT No.: PCT/EP2007/009382

§ 371 Date: Mar. 25, 2009

(30) Foreign Application Priority Data
Oct. 30, 2006 (EP) 06123156.9

(51) Int. Cl. A61K 31/5025; C07D 487/00

(52) U.S. Cl. 514/248; 544/236

(57) ABSTRACT

A compound of Formula Ia or Ib in free or salt or solvate form, where R¹, R², R³, R⁴, R⁵ R²⁰, R²⁴, R²⁵, X, Y and Z have the meanings as indicated in the specification, are useful for treating diseases mediated by the ALK-5 and/or ALK-4 receptor. These compounds are also useful for treating diseases mediated by the Pi3k receptor, the JAK-2 receptor and the TRK receptor. Pharmaceutical compositions that contain the compounds and processes for preparing the compounds are also described.

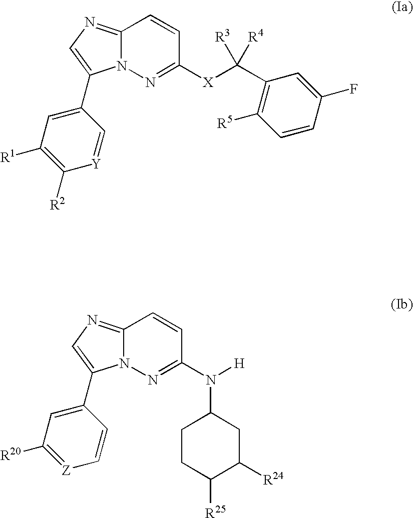

This invention relates to organic compounds and their use as pharmaceuticals, in particular for the treatment of inflammatory or obstructive airways diseases such as pulmonary hypertension, pulmonary fiborosis, liver fibrosis; cancer; muscle diseases such as muscle atrophies and muscle dystrophies and systemic skeletal disorders such as osteoporosis.

In one aspect, the present invention provides a compound of Formula Ia or Ib

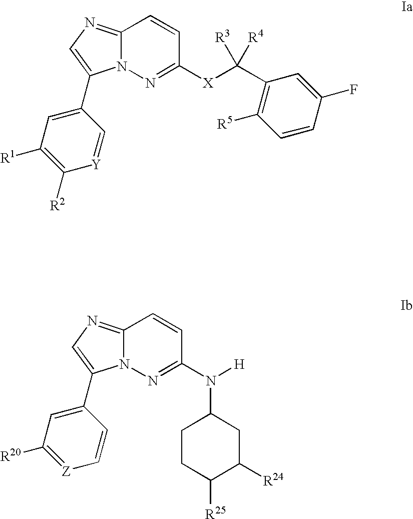

in free or salt or solvate form, wherein:

### X is O or NH;

### Y is CR¹³ or N;

R¹ is selected from H, CN, halo, —C(O)NR⁷R⁸ and

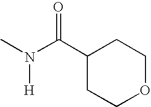

R² is selected from H, CN, morpholino, tetrazole optionally substituted by C₁-C₃ alkyl, —S(O)₂NH₂, —C(O)NR⁷R⁸ and CH₂OH,
provided that R¹ and R² are not both H and provided that when R² is other than H, R¹ is H or halo; and when R¹ is other than H, R² is H; or R¹ and R² together with the carbon atoms to which they are attached form a 6-membered heterocyclic ring containing at least one heteroatom selected from N, O and S, the heterocyclic ring being optionally substituted by C₁-C₃ alkyl or an oxo group;
R³ is selected from H, Me and CH₂OH;
R⁴ is H or C₁-C₃ alkyl;
R⁵ is H or halogen;
R⁷ is H or C₁-C₃ alkyl;
R⁸ is independently selected from H, C₁-C₆ alkyl, (CH₂)ₘhet and (CH₂)ₙNR⁹R¹⁰; or
R⁷ and R⁸ together with the nitrogen atom to which they are attached form a 5- or 6-membered heterocyclic ring optionally containing a further heteroatom selected from N, O and S, the ring being optionally substituted by C₁-C₃ alkyl or NR¹¹R¹²;
R⁹, R¹⁰, R¹¹ and R¹² are each independently selected from H and C₁-C₃ alkyl;
R¹³ is H or halo;
m and n are each independently 0, 1 or 2;
het is a 5- or 6-membered heterocyclic ring containing one or two heteroatoms selected from N, O and S, the ring being optionally substituted by C₁-C₃ alkyl;

### Z is N or CR²⁶;

R²⁰ is selected from H, cyclopropyl and R²¹, provided that when Z is N, R²⁰ is other than H;
R²¹ is selected from

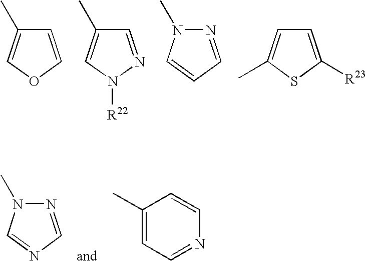

R²² and R²³ are each independently selected from H and C₁-C₃ alkyl;
R²⁴ is selected from H and OH;
R²³ is selected from H, OH and CH₂OH; provided that when R²⁴ is H, R²⁵ is OH or CH₂OH; and when R²⁴ is OH, R²⁵ is H; and
R²⁶ is selected from H and R²¹, provided that when R²⁰ is other than H, R²⁶ is H; and when R²⁰ is H, R²⁶ is R²¹.

Terms used in the specification have the following meanings:

“Optionally substituted at one, two or three positions” as used herein means the group referred to can be substituted at one or two or three positions by any one or any combination of the radicals listed thereafter.
“Halo” or “halogen” as used herein denotes a element belonging to group 17 (formerly group VII) of the Periodic Table of Elements, which may be, for example, fluorine, chlorine, bromine or iodine.
“C₁-C₈-alkyl” as used herein denotes straight chain or branched alkyl that contains one to eight carbon atoms. If a different number of carbon atoms is specified, for example C₆ or C₃, then the definition is to be amended correspondingly.
“4-, 5-, or 6-membered heterocyclic group”, refers to a 4-, 5- or 6-membered heterocyclic ring containing at least one ring heteroatom selected from the group consisting of nitrogen, oxygen and sulphur, which may be saturated or partially saturated. Examples of such heterocyclic groups include but are not limited to azetidine, pyrrolidine, pyrroline, piperidine, piperazine, pyrrolidinone, morpholine, oxazine, tetrahydrofuran, tetrahydrothiophene, tetrahydrothiopyran, tetrahydropyran, 1,4-dioxane and 1,4-oxathiane, The heterocyclic group can be unsubstituted or substituted.
“C₃-C₁₀-cycloalkyl” denotes a fully saturated carbocyclic ring having 3 to 10 ring carbon atoms, for example a monocyclic group such as a cyclopropyl, cyclobutyl, cyclopentyl or cyclohexyl, cycloheptyl, cyclooctyl, cyclononyl or cyclodecyl, or a bicyclic group such as bicycloheptyl or bicyclooctyl. If a different number of carbon atoms is specified, for example C₆ or C₈, then the definition is to be amended correspondingly.
“C₁-C₈-haloalkyl” as used herein denotes C₁-C₈-alkyl as hereinbefore defined substituted by one or more halogen atoms, preferably one, two or three halogen atoms. If a different number of carbon atoms is specified, for example C₆ or C₃, then the definition is to be amended correspondingly.
“C₁-C₈-alkylamino” and “di(C₁-C₈-alkyl)amino” as used herein denote amino substituted respectively by one or two C₁-C₈-alkyl groups as hereinbefore defined, which may be the same or different. If a different number of carbon atoms is specified, for example C₆ or C₃, then the definition is to be amended correspondingly.
“C₁-C₈-alkoxy” as used herein denotes straight chain or branched alkoxy that contains 1 to 8 carbon atoms. If a different number of carbon atoms is specified, for example C₆ or C₃, then the definition is to be amended correspondingly.
Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

In an embodiment of the present invention as defined anywhere above, R¹ is selected from H, CN, halo,

—C(O)NR⁷R⁸ and

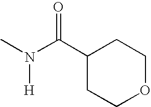

R² is selected from H, CN, morpholino, tetrazole optionally substituted by C₁-C₃ alkyl, —S(O)₂NH₂, —C(O)NR⁷R⁸ and CH₂OH,
provided that R¹ and R² are not both H and provided that when R² is other than H, R¹ is H; and when R¹ is other than H, R² is H.
In an embodiment of the present invention as defined anywhere above, R⁴ is H or Me.
In an embodiment of the present invention as defined anywhere above, R⁵ is H or F.
In an embodiment of the present invention as defined anywhere above, R⁷ is H or Me.
In an embodiment of the present invention as defined anywhere above, R¹³ is H.
In an embodiment of the present invention as defined anywhere above, R²⁴ is H.
In a further embodiment of the invention, the compound according to Formula Ia or Ib is selected from:
4-(3-[2,4′]Bipyridinyl-4-yl-imidazo[1,2-b]pyridazin-6-ylamino)-cyclohexanol;
4-(3-[2-(5-Methyl-thiophen-2-yl)-pyridin-4-yl]-imidazo[1,2-b]pyridazin-6-ylamino)-cyclohexanol;
4-[3-(2-Furan-3-yl-pyridin-4-yl)-imidazo[1,2-b]pyridazin-6-ylamino]-cyclohexanol;
4-(3-[2-(1-Methyl-1H-pyrazol-4-yl)-pyridin-4-yl]-imidazo[1,2-b]pyridazin-6-ylamino)-cyclohexanol;
4-[3-(4-Pyrazol-1-yl-phenyl)-imidazo[1,2-b]pyridazin-6-ylamino]-cyclohexanol;
4-[3-(2-Cyclopropyl-pyridin-4-yl)-imidazo[1,2-b]pyridazin-6-ylamino]-cyclohexanol;
4-[3-(3-Pyrazol-1-yl-phenyl)-imidazo[1,2-b]pyridazin-6-ylamino]-cyclohexanol;
4-[3-(4-[1,2,4]Triazol-1-yl-phenyl)-imidazo[1,2-b]pyridazin-6-ylamino]-cyclohexanol;
(4-[3-(4-Pyrazol-1-yl-phenyl)-imidazo[1,2-b]pyridazin-6-ylamino]-cyclohexyl)-methanol;
(4-[6-(2,5-Difluoro-benzylamino)-imidazo[1,2-b]pyridazin-3-yl]-phenyl)-(4-methyl-piperazin-1-yl)-methanone;
4-[6-(2,5-Difluoro-benzylamino)-imidazo[1,2-b]pyridazin-3-yl]-N-(2-morpholin-4-yl-ethyl)-benzamide;
{4-[6-(2,5-Difluoro-benzylamino)-imidazo[1,2-b]pyridazin-3-yl]-phenyl}-(4-dimethylamino-piperidin-1-yl)-methanone;
{4-[6-(2,5-Difluoro-benzylamino)-imidazo[1,2-b]pyridazin-3-yl]-phenyl}-morpholin-4-yl-methanone;
(3-[6-(2,5-Difluoro-benzylamino)-imidazo[1,2-b]pyridazin-3-yl]-N-(tetrahydro-pyran-4-yl)-benzamide;
4-[6-(2,5-Difluoro-benzylamino)-imidazo[1,2-b]pyridazin-3-yl]-N-(1-ethyl-pyrrolidin-2-ylmethyl)-benzamide;
4-[6-(2,5-Difluoro-benzylamino)-imidazo[1,2-b]pyridazin-3-yl]-N-(tetrahydro-pyran-4-yl)-benzamide;
4-[6-(3-Fluoro-benzylamino)-imidazo[1,2-b]pyridazin-3-yl]-benzenesulfonamide;
4-[6-(3-Fluoro-benzylamino)-imidazo[1,2-b]pyridazin-3-yl]-benzamide;
4-(6-[(R or S)-1-(3-Fluoro-phenyl)-2-hydroxy-ethylamino]-imidazo[1,2-b]pyridazin-3-yl)-benzonitrile;
3-(6-[(R)-1-(3-Fluoro-phenyl)-ethylamino]-imidazo[1,2-b]pyridazin-3-yl)-benzonitrile;
{4-[6-(3-Fluoro-benzyloxy)-imidazo[1,2-b]pyridazin-3-yl]-phenyl}-methanol; and
Tetrahydro-pyran-4-carboxylic acid {3-[6-(2,5-difluoro-benzylamino)-imidazo[1,2-b]pyridazin-3-yl]-phenyl}-amide.

According to Formula Ia or Ib, the embodiments of the invention as defined anywhere herein may be incorporated independently, collectively or in any combination.

Compounds of Formula Ia or Ib that contain a basic centre are capable of forming acid addition salts, particularly pharmaceutically acceptable acid addition salts. Pharmaceutically acceptable acid addition salts of the compound of Formula Ia or Ib include those of inorganic acids, for example, hydrohalic acids such as hydrofluoric acid, hydrochloric acid, hydrobromic acid, hydroiodic acid, nitric acid, sulfuric acid, phosphoric acid; and organic acids, for example aliphatic monocarboxylic acids such as formic acid, acetic acid, trifluoroacetic acid, propionic acid and butyric acid, caprylic acid, dichloroacetic acid, hippuric acid, aliphatic hydroxy acids such as lactic acid, citric acid, tartaric acid or malic acid, gluconic acid, mandelic acid, dicarboxylic acids such as maleic acid or succinic acid, adipic acid, aspartic acid, fumaric acid, glutamic acid, malonic acid, sebacic acid, aromatic carboxylic acids such as benzoic acid, p-chloro-benzoic acid, nicotinic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid, ethanesulfonic acid, ethane-1,2-disulfonic acid, 2-hydroxy-ethanesulfonic acid, (+) camphor-10-sulfonic acid, naphthalene-2-sulfonic acid, naphthalene-1,5-disulfonic acid or p-toluenesulfonic acid. These salts may be prepared from compounds of Formula Ia or Ib by known salt-forming procedures. Pharmaceutically acceptable solvates are generally hydrates.

Compounds of Formula Ia or Ib which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine, arginine, benethamine, benzathine, diethanolamine, 4-(2-hydroxy-ethyl)morpholine, 1-(2-hydroxyethyl)pyrrolidine, N-methyl glutamine, piperazine, triethanol-amine or tromethamine. These salts may be prepared from compounds of formula I by known salt-forming procedures. Compounds of Formula Ia or Ib that contain acidic, e.g. carboxyl, groups may also exist as zwitterions with the quaternary ammonium centre.

Compounds of Formula Ia or Ib in free form may be converted into salt form, and vice versa, in a conventional manner. The compounds in free or salt form can be obtained in the form of hydrates or solvates containing a solvent used for crystallisation. Compounds of Formula Ia or Ib can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Many compounds of the invention contain at least one asymmetric carbon atom and thus they exist in individual optically active isomeric forms or as mixtures thereof, e.g. as racemic mixtures. In cases where additional asymmetric centres exist the present invention also embraces both individual optically active isomers as well as mixtures, e.g. diastereomeric mixtures, thereof.

The invention includes all such forms, in particular the pure isomeric forms. The different isomeric forms may be separated or resolved one from the other by conventional methods, or any given isomer may be obtained by conventional synthetic methods or; by stereospecific or asymmetric syntheses. Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

The invention includes all pharmaceutically acceptable isotopically-labelled compounds of Formula Ia or Ib wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen e.g. ²H and ³H, carbon e.g. ¹¹C, ¹³C and ¹⁴C, chlorine e.g. ³⁶Cl, fluorine e.g. ¹⁸F, iodine e.g. ¹²³I and ¹²⁵I, nitrogen e.g. ¹³N and ¹⁵N, oxygen e.g. ¹⁵O, ¹⁷O and ¹⁸O, and sulfur e.g. ³⁵S.

Certain isotopically-labelled compounds of Formula Ia or Ib, for example those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with heavier isotopes such as deuterium (²H) may afford certain therapeutic advantages that result from greater metabolic stability, for example increased in vivo half-life or reduced dosage requirements, and hence may be preferred in some circumstances. Substitution with positron emitting isotopes, such as ¹¹C, ¹⁸F, ¹⁵O, and ¹³N can be useful in Positron Emission Topography (PET) studies for examining substrate receptor occupancy.

Isotopically-labelled compounds of Formula Ia or Ib can generally be prepared by conventional techniques known to those skilled in the art or by processes analogous to those described in the accompanying examples using an appropriate isotopically-labelled reagent in place of the non-labelled reagent previously used.

Pharmaceutically acceptable solvates in accordance with the invention include those wherein the solvent of crystallisation may be isotopically substituted e.g. D₂O, d₆-acetone or d₆-DMSO.

Specific especially preferred compounds of the invention are those described hereinafter in the Examples.

The present invention also provides a process for the preparation of compounds of Formula Ia and Ib in free or salt or solvate form. They can be prepared by a process comprising:

(i) (A) reacting a compound of formula IIa

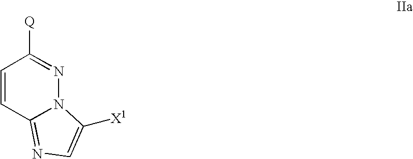

where Q represents the relevant groups as defined in Formula Ia and Ib above, namely:

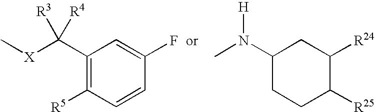

where X, R³, R⁴, R⁵, R²⁴ and R²⁵ are as defined anywhere above, and X¹ is halo, with a compound of formula IIIa or IIIb

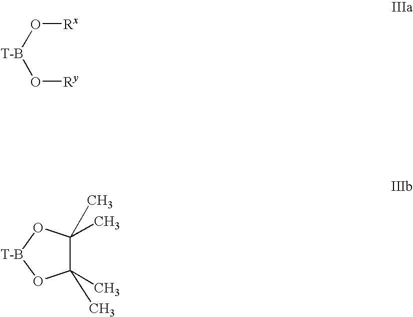

where T represents the relevant groups as defined in Formula Ia and Ib above, namely:

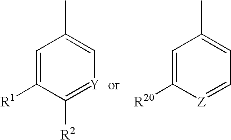

where Y, Z, R¹, R² and R²⁰ are as defined anywhere above, and R^{x} and R^{y} are independently hydrogen or C₁-C₈-alkyl;
(B) for the preparation of compounds of Formula Ia or Ib where Q includes a nitrogen linking group, reacting a compound of formula IV

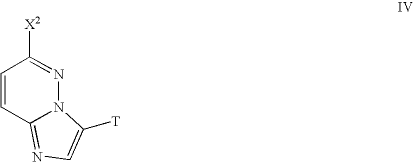

where T is as hereinbefore defined and X² is halo, with a compound of formula V

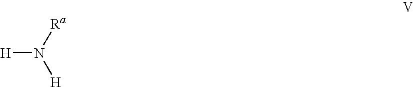

where R^{a} represent the relevant groups as defined in Formula Ia and Ib above, namely:

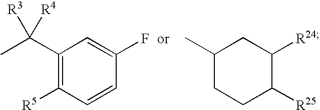

and where R³, R⁴, R⁵, R²⁴ and R²⁵ are as defined anywhere above;
(C) for the preparation of compounds of Formula Ia or Ib where Q includes a nitrogen linking group or an oxygen linking group and T is as defined above, reacting a compound of formula VI

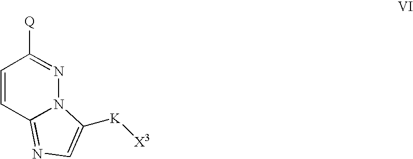

where Q is as hereinbefore defined, K is a 6-membered heteroaromatic group and X³ is halo, with a compound of formula VIIa or VIIb

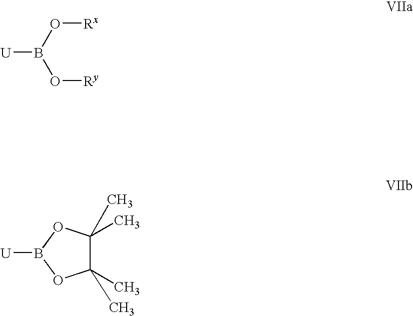

where U is —R¹, —R² or —R²⁰ and R^{x} and R^{y} are independently hydrogen or C₁-C₈-alkyl; or
(D) for the preparation of compounds of Formula Ia where Q includes an oxygen linking group, reacting a compound of formula IV where T is as hereinbefore defined and X² is halo, with a compound of formula VIII

HO—R^{c} VIII
where R^{c} is a substituted benzyl group in accordance with the compounds defined by Formula Ia; and
   (ii) recovering the resultant compound of Formula Ia or Ib in free or salt or solvate form.

Process variant (A) may be carried out using known procedures for reacting halogenated heterocyclic groups with aryl/heteroaryl boronic acids or analogously as hereinafter described in the Examples. The reaction is conveniently carried out in an organic solvent, for example a mixture of dioxane and water, preferably in the presence of a catalyst e.g. palladium dichloridebistriphenylphosphine, and an inorganic base e.g. sodium carbonate. Suitable reaction temperatures are elevated temperatures, e.g. from 100° C. to 150° C., preferably by microwaving at about 100° C., e.g. for about 120 minutes.

Process variant (B) may be carried out using known procedures for reacting halides, especially halo-substituted heterocyclic compounds, with amines, or analogously as hereinafter described in the Examples. The reaction is conveniently carried out using an organic solvent, for example N-methyl-pyrrolidinone (NMP) optionally in the presence of an inorganic base e.g. sodium carbonate. Suitable reaction temperatures are from 100° C. to 250° C., preferably between 120° C. to 220° C., especially about 180° C., for example by heating with microwave radiation, e.g. for about 90 minutes.

Process variant (C) may be carried out using known procedures for reacting halogenated heterocyclic groups with aryl/heteroaryl boronic acids or analogously as hereinafter described in the Examples. The reaction is conveniently carried out in an organic solvent, for example a mixture of dioxane and water, preferably in the presence of a catalyst e.g. palladium dichloridebistriphenylphosphine, and an inorganic base e.g. sodium carbonate. Suitable reaction temperatures are elevated temperatures, e.g. from 100° C. to 150° C., preferably with microwave radiation at about 120° C., e.g. for about 120 minutes.

Process variant (D) may be carried out using known procedures for reacting halides, especially halogenated heterocyclic groups, with primary alcohols or analogously as hereinafter described in the Examples. The reaction is conveniently carried out in an organic solvent, for example dimethylformamide, preferably in the presence of a base e.g. sodium hydride. Suitable reaction temperatures are from 10° C. to 40° C., but preferably room temperature.

Compounds of formula IIa are prepared by reacting a compound of formula IX

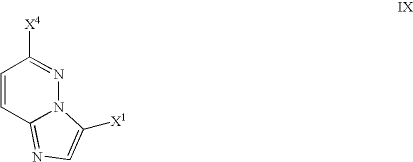

where X¹ and X⁴ are each halo, with a compound of formula X

Q—H X

where Q is as hereinbefore defined or analogously as hereinafter described in the Examples.

The reaction is conveniently carried out in an organic solvent, for example N-methyl-2-pyrrolidone (NMP), preferably in the presence of an inorganic base e.g. sodium bicarbonate (NaHCO₃). Suitable reaction temperatures are elevated temperatures, e.g. from 100° C. to 200° C., preferably with microwave radiation at about 180° C., e.g. for about 40 minutes.

Compounds of formulae IIIa or IIIb are commercially available or may be prepared by known methods.

Compounds of formula IV are prepared by reacting a compound of formula XI

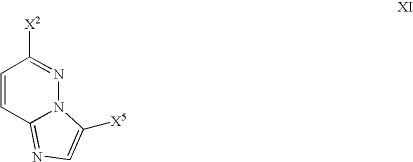

where X² and X⁵ are each halo, with a compound of formula IIIa or IIIb

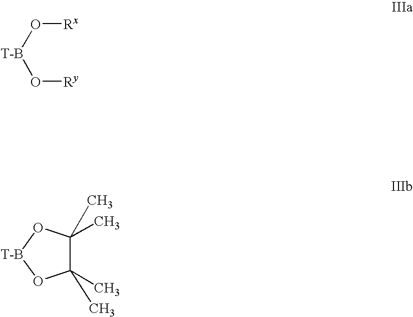

where T is as hereinbefore defined and R^{x} and R^{y} are independently hydrogen or C₁-C₈-alkyl or analogously as hereinafter described in the Examples. The reaction is conveniently carried out in an organic solvent, for example a mixture of dioxane and water, preferably in the presence of a catalyst e.g. palladium dichloridebistriphenylphosphine, and an inorganic base e.g. sodium carbonate. Suitable reaction temperatures are elevated temperatures, e.g. from 100° C. to 150° C., preferably by microwaving at about 100° C., e.g. for about 120 minutes.

Compounds of formula V are commercially available or may be prepared by known methods.

Compounds of formula VI are prepared by reacting a compound of formula Ia where Q is as hereinbefore defined and X¹ is halo, with a compound of formula XIIa or XIIb

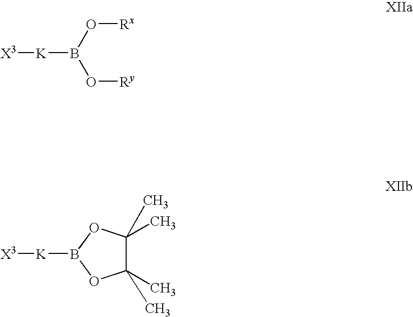

where X³ is halo, K is as defined above and R^{x} and R^{y} are independently hydrogen or C₁-C₈-alkyl, or analogously as hereinafter described in the Examples. The reaction is conveniently carried out in an organic solvent, for example a mixture of dioxane and water, preferably in the presence of a catalyst e.g. palladium dichloridebistriphenylphosphine, and an inorganic base e.g. sodium carbonate. Suitable reaction temperatures are elevated temperatures, e.g. from 100° C. to 150° C., preferably by microwaving at about 100° C., e.g. for about 120 minutes.

Compounds of formulae VIIa or VIIb, VIII, IX, X, XI, XIIa or XIIb are commercially available or may be prepared by known methods.

Compounds of Formula Ia and Ib in pharmaceutically acceptable salt form are hereinafter referred to as “agents of the invention”. These compounds are useful as pharmaceuticals.

The agents of the invention act as activin-like kinase (“ALK”)-5 inhibitors. At least many of these compounds also act as ALK-4 inhibitors too.

TGF-β1 is the prototypic member of a family of cytokines including the TGF-βs, activins, inhibins, bone morphogenetic proteins and Mullerian-inhibiting substance, that signal through a family of single transmembrane serine/threonine kinase receptors. These receptors can be divided into two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signalling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor.

Activins transduce signals in a manner similar to TGF-β. Activins bind to serine/threonine kinase, the activin type II receptor (ActRIIB), and the activated type II receptor hyper-phosphorylates serine/threonine residues in the GS region of the ALK4. The activated ALK4 in turn phosphorylates Smad2 and Smad3. The consequent formation of a hetero-Smad complex with Smad4 results in the activin-induced regulation of gene transcription.

Activation of the TGF-β1 axis and expansion of extracellular matrix are early and persistent contributors to the development and progression of chronic renal disease and vascular disease. Border W. A., et al, N. Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818, allograft rejection, HIV nephropathy and angiotensin-induced nephropathy Border W. A., et al, N. Engl. 5 J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGFβ1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991 1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-I variant cells would be allowed to grow in a slow, but uncontrolled fashion, while overproducing extracellular matrix components McCaffrey T. A., et al, Jr., J. Clin.; Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodelling via a TGF-β-dependent mechanism. Therefore, inhibiting the action of TGF-β1 on ALK5 is also indicated in atherosclerosis and restenosis.

Liver fibrosis is the result of unbalanced wound healing response to chronic liver injury trigged by a number of agents, such as hepatitis B and hepatitis C virus, alcohol or drugs, and autoimmune diseases. Ultimately, liver fibrosis could lead to life-threatening cirrhosis and liver cancer (see review article by Gressner et al (2006)J. Cell. Mol. Med. 2006, 10(1): 76-99).

Several cellular signaling pathways are known to be altered upon chronic liver injury. TGFβ signaling, its receptors and associated Smad-signaling proteins are well documented to be present in cell types involved in fibrogenesis. The circulating levels of TGFβ have been found to be elevated in a number of animal models of fibrotic diseases including liver fibrosis. Transgenic mice with overexpression of TGFβ1 develop fibrosis in multiple organs including liver, kidney, lungs and heart. It is apparent that an elevated TGFβ signaling is involved in all types of fibrotic diseases including liver fibrosis. This notion has been further validated in several studies using TGFβ inhibitors in fibrosis models. TGFβ mediates it signal by binding to two ser/thr kinase receptors, TGFβRII and ALK5. Expressing a dominant negative TGFβRII showed beneficial effects in a rat model of dimethylnitrosamine induced liver fibrosis (see Qi et al (1999) Proc. Natl. Acad. Sci. 96: 2345-9 and Nakamura et al (2000) Hepatology 32: 247-55). Inhibiting TGFβ expression using an antisense approach also reduced liver fibrosis induced by bile duct ligation (see Arias et al (2003) BMC Gastroenterol. 3: 29). Recently, a small molecule inhibitor of ALK5, GW6604, when given therapeutically to rat, had significant effect in the treatment of dimethylnitrosamine induced liver fibrosis. It is quite remarkable that GW6604 prevented 40% of the death rate and inhibited extracellular matrix deposition by 60%, a key measurement for fibrosis. Importantly, no obvious side effects were noted during the 3 weeks treatment with GW6604 (see De Gouville et al (2005) Br. J. Pharmacol. 145: 166-77). Taken together these studies suggest that inhibiting TGFβ signaling could be an effective treatment for liver fibrotic diseases.

TGF-β1 is also indicated in wound repair. Neutralizing antibodies to TGF-β1 have been used in a number of models to illustrate that inhibition of TGF-β1 signalling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signalling pathways.

TGF-β is also implicated in peritoneal adhesions Sand G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

TGF-β is also implicated in photoaging of the skin (see Fisher G J. Kang S W. Varani J. Bata-Csorgo Z.Wan Y S. Data S. Voorhees J J., Mechanisms of photoaging and chronological skin ageing, Archives of Dermatology, 138(11):1462-1470, 2002 November and Schwartz E. Sapadin A N. Kligman L H. “Ultraviolet B radiation increases steady state mRNA levels for cytokines and integrins in hairless mouse skin-modulation by 25 topical tretinoin”, Archives of Dermatological Research, 290(3):137-144, 1998 Mar.)

TGF-β signalling is also implicated in the development of pulmonary disorders, in particular pulmonary hypertension and pulmonary fibrosis (see Morrell N R, Yang X, Upton P D, Jourdan K B, Morgan N, Sheares K K, Trembath R C., Altered growth responses of pulmonary artery smooth muscle cells from patients with primary pulmonary hypertension to transforming growth factor-beta(1) and bone morphogenetic proteins. Circulation. 2001 Aug. 14; 104(7):790-5. Bhatt N, Baran C P, Allen J, Magro C, Marsh C B., Promising pharmacologic innovations in treating pulmonary fibrosis. Curr Opin Pharmacol. 2006 Apr. 28).

TGF-β1 levels are increased in animal models of pulmonary hypertension (Mata-Greenwood E, Meyrick B, Steinhorn R H, Fineman J R, Black S M. Alterations in TGF-beta1 expression in lambs with increased pulmonary blood flow and pulmonary hypertension. Am. J. Physiol. Lung Cell Mol. Physiol. 2003 July; 285(1):L209-21). Other studies have suggested that pulmonary endothelial cell-derived TGF-β1 can stimulate the growth of pulmonary vascular smooth muscle cells which may underlie the enhanced muscularisation observed in the pulmonary vasculature of individuals with pulmonary hypertension (Sakao S, Taraseviciene-Stewart L, Wood K, Cool C D, Norbert V F. Apoptosis of pulmonary microvascular endothelial cells stimulates vascular smooth muscle cell growth. Am. J. Physiol. Lung Cell Mol. Physiol. 2006 Apr. 14). Therefore, inhibiting the action of TGF-β1 on ALK5 is indicated as a therapeutic intervention in pulmonary hypertension.

Additionally, dys-regulated TGF-β signalling has also been implicated in the development of idiopathic pulmonary fibrosis. Activation of ALK5 results in Smad3-activation and downstream modulation of the expression of genes involved in the fibrotic process such as plasminogen activator inhibitor-1, pro-collagen 3A1, and connective tissue growth factor. The levels of TGF-β1 and its downstream pro-fibrotic mediators have been demonstrated to be up-regulated in bronchoalveolar lavage taken from patients with idiopathic pulmonary fibrosis (Hiwatari N, Shimura S, Yamauchi K, Nara M, Hida W, Shirato K. Significance of elevated procollagen-III-peptide and transforming growth factor-beta levels of bronchoalveolar lavage fluids from idiopathic pulmonary fibrosis patients. Tohoku J. Exp. Med. 1997 February; 181(2): 285-95) and in animal models of idiopathic pulmonary fibrosis (Westergren-Thorsson G, Hernnas J, Sarnstrand B, Oldberg A, Heinegard D, Malmstrom A. Altered expression of small proteoglycans, collagen, and transforming growth factor-beta 1 in developing bleomycin-induced pulmonary fibrosis in rats. J. Clin. Invest. 1993 August; 92(2):632-7).

Transient over-expression of active TGF-β1 in murine lungs, using adenoviral vector-mediated gene transfer, resulted in progressive pulmonary fibrosis in wild-type mice, whereas no fibrosis was seen in the lungs of Smad3 knockout mice up to 28 days following TGF-β1 challenge (Khalil N, Parekh T V, O'Connor R N, Gold L I. Differential expression of transforming growth factor-beta type I and II receptors by pulmonary cells in bleomycin-induced lung injury: correlation with repair and fibrosis. Exp. Lung. Res. 2002 April-May; 28(3):233-50. Thus, inhibition of TGF-β1 activation of ALK5 is also indicated for pulmonary fibrosis.

Activin signalling and overexpression of activin is linked to pathological disorders that involve extracellular matrix accumulation and fibrosis (e.g., Matsuse, T. et al., Am. J. Respir Cell Mol. Biol. 13:17-24 (1995); Inoue, S. et al., Biochem. Biophys. Res. Comn. 205:441-448 (1994); Matsuse, T. et al., Am. J. Pathol. 148:707-713 (1996); De Bleser et al., Hepatology 26:905-912 (1997); Pawlowski, J. E., et al., J. Clin. Invest. 100:639-648 (1997); Sugiyama, M. et al., Gastroenterology 114:550-558 (1998); Munz, B. et al., EMBO J. 18:5205-5215 (1999)), inflammatory responses (e.g., Rosendahl, A. et al., Am. J. Respir. Cell Mol. Biol. 25:60-68 (2001), cachexia or wasting (Matzuk7 M. M. et al., Proc. Natl. Acad. Sci. USA 91:8817-8821 (1994); Coerver, K. A. et al., Mol. Endocrinol. 10:531 543 (1996); Cipriano, S. C. et al., Endocrinology 141:2319-2327 (2000)), diseases or pathological responses in the central nervous system (e.g., Logan, A. et al., Eur. J. Neurosci. 11:2367-2374 (1999); Logan, A. et al., Exp. Neurol. 159:504-510 (1999); Masliah, E. et al., Neurochem. Int. 39:393-400 (2001); De Groot, C. J. A. et al., J. Neuropathol. Exp. Neural. 58:174-187 (1999); John, G. R. et al., Nat. Med. 8:1115-1121 (2002)) and hypertension (e.g., Dahly, A. J. et al., Am. J. Physiol. Regul. Integr Comp. Physiol. 283: R757-767 (2002)). Studies have shown that TGF-β and activin can act synergistically to induce extracellular matrix production (e.g., Sugiyama, M. et al., Gastroerterology 114; 550-558 (1998)).

It follows, therefore, that inhibition of ALK5 and/or ALK4 phosphorylation of Smad2 and Smad3 by the agents of the invention can be useful to treat and prevent disorders that involve these signalling pathways.

Activin signalling is also implicated in the development of pulmonary disorders, in particular pulmonary hypertension and pulmonary fibrosis. For example, the expression of activin A in lung samples from patients with interstitial pulmonary fibrosis demonstrated strong expression of activin A on metaplastic epithelium, hyperplastic smooth muscle cells, desquamated cells, and alveolar macrophages. Pulmonary arteries from patients with primary or secondary pulmonary hypertension showed abundant immunoreactive activin A on smooth muscle cells. These findings suggest a potential role for this growth factor, activin A, in the pathogenesis of pulmonary tissue remodelling associated with interstitial pulmonary fibrosis and pulmonary hypertension (Matsuse T, Ikegami A, Ohga E, Hosoi T, Oka T, Kida K, Fukayama M, Inoue S, Nagase T. Ouchi Y, Fukuchi Y. Expression of immunoreactive activin A protein in remodelling lesions associated with interstitial pulmonary fibrosis. Am. J. Pathol. 1996 March; 148(3):707-13). An increase in fibroblasts and associated connective tissue is a feature of pulmonary fibrosis and pulmonary hypertension. Activin A has been demonstrated to modulate human lung fibroblast (HFL1) activity, particularly with respect to proliferation and its differentiation into myofibroblast, thus activin A has potential effects on proliferation of lung fibroblast and its differentiation into myofibroblast, and may contribute to structural remodelling observed in pulmonary fibrosis and hypertension (Ohga E, Matsuse T, Teramoto S, Katayama H, Nagase T, Fukuchi Y, Ouchi Y. Effects of activin A on proliferation and differentiation of human lung fibroblasts. Biochem. Biophys. Res. Commun. 1996 Nov. 12; 228(2):391-6). The induction of pulmonary fibrosis mediated by bleomycin challenge in rats results in the up-regulated expression of activin A in macrophages infiltrated in the lung, and was detected in fibroblasts accumulated in the fibrotic area. Administration of follistatin, an antagonist of activin signalling to bleomycin-treated rats significantly reduced the number of macrophages and neutrophils in bronchoalveolar lavage and reduced the protein content. Follistatin markedly reduced the number of infiltrating cells, ameliorated the destruction of lung architecture, and attenuated lung fibrosis (Aoki F, Kurabayashi M, Hasegawa Y, Kojima 1. Attenuation of bleomycin-induced pulmonary fibrosis by follistatin. Am. J. Respir. Crit. Care Med. 2005 Sep. 15; 172(6):713-20).

Therefore, inhibiting activin signalling via ALK4 inhibition may also be beneficial for the treatment of pulmonary fibrosis and pulmonary hypertension.

It has been demonstrated recently that reduction in TGF-β signalling, through its effector Smad3, enhances the mechanical properties and mineral concentration of the bone matrix, as well as the bone mass, enabling the bone to better resist fracture. These results suggest that reduction of TGF-β signalling could be considered as a therapeutic target to treat bone disorders. (Balooch G, et al. Proc. Natl. Acad. Sci. USA. 2005 Dec. 27; 102(52):18813-8). Thus, inhibition of TGF-β1 activation of ALK5 is also indicated for increasing mineral density strength and content of bone and may be utilized to treat a wide variety of conditions, including for example, osteopenia, osteoporosis, fractures and other disorders in which low bone mineral density are a hallmark of the disease.

Having regard to their inhibition of ALK-5 and/or ALK4 receptors, agents of the invention are useful in the treatment of conditions mediated by the ALK-5 and/or ALK4 receptors. Treatment in accordance with the invention may be symptomatic or prophylactic.

Therefore according to a further aspect, the invention provides the use of agents of the invention in the preparation of a medicament for treating or preventing a disease or condition mediated by ALK-5 inhibition or ALK-4 inhibition.

Diseases or condition mediated by ALK-5 inhibition or ALK4 inhibition include glomerulo-nephritis, diabetic nephropathy, lupus nephritis, hypertension-induced nephropathy, renal interstitial fibrosis, renal fibrosis resulting from complications of drug exposure, HIV-associated nephropathy, transplant necropathy, liver fibrosis due to all etiologies, hepatic dysfunction attributable to infections, alcohol-induced hepatitis, disorders of the biliary tree, pulmonary fibrosis, pulmonary hypertension, acute lung injury, adult respiratory distress syndrome, idiopathic pulmonary fibrosis, chronic obstructive pulmonary disease, pulmonary disease due to infectious or toxic agents, post-infarction cardiac fibrosis, congestive heart failure, dilated cardiomyopathy, myocarditis, vascular stenosis, restenosis, atherosclerosis, ocular scarring, corneal scarring, proliferative vitreoretinopathy, excessive or hypertrophic scar or keloid formation in the dermis occurring during wound healing resulting from trauma or surgical wounds, peritoneal and sub dermal adhesion, scleroderma, fibrosclerosis, progressive systemic sclerosis, dermatomyositis, polymyositis, arthritis, ulcers, impaired neurological function, male erectile dysfunction, Alzheimer's disease, Raynaud's syndrome, fibrotic cancers, tumor metastasis growth, radiation-induced fibrosis, thrombosis, and bone conditions such as osteopenia and osteoporosis, which are associated with increased calcium depletion or resorption or in which stimulation of bone formation and calcium fixation in the bone is desirable.

Diseases or conditions mediated by ALK-5 inhibition in particular include chronic renal disease, acute renal disease, wound healing, arthritis, osteoporosis, kidney disease, congestive heart failure, inflammatory or obstructive airways diseases, pulmonary hypertension, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photaging of the skin.

Inflammatory or obstructive airways diseases to which the present invention is applicable include asthma of whatever type or genesis including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant from any previously administered symptomatic asthma therapy.

Other inflammatory or obstructive airways diseases and conditions to which the present invention is applicable include adult/acute respiratory distress syndrome (ARDS), chronic obstructive pulmonary or airways disease (COPD or COAD), including chronic bronchitis, or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Preferably the disease or condition mediated by ALK-5 inhibition or ALK-4 inhibition is pulmonary hypertension, pulmonary fibrosis, liver fibrosis or osteoporosis.

Pulmonary hypertension to be treated in accordance with the invention includes primary pulmonary hypertension (PPH); secondary pulmonary hypertension (SPH); familial PPH; sporadic PPH; precapillary pulmonary hypertension; pulmonary arterial hypertension (PAH); pulmonary artery hypertension; idiopathic pulmonary hypertension; thrombotic pulmonary arteriopathy (TPA); plexogenic pulmonary arteriopathy; functional classes I to IV pulmonary hypertension; and pulmonary hypertension associated with, related to, or secondary to, left ventricular dysfunction, mitral valvular disease, constrictive pericarditis, aortic stenosis, cardiomyopathy, mediastinal fibrosis, anomalous pulmonary venous drainage, pulmonary venoocclusive disease, collagen vascular disease, congenital heart disease, HIV virus infection, drugs and toxins such as fenfluramines, congenital heart disease, pulmonary venous hypertension, chronic obstructive pulmonary disease, interstitial lung disease, sleep-disordered breathing, alveolar hypoventilation disorder, chronic exposure to high altitude, neonatal lung disease, alveolar-capillary dysplasia, sickle cell disease, other coagulation disorder, chronic thromboemboli, connective tissue disease, lupus, schistosomiasis, sarcoidosis or pulmonary capillary hemangiomatosis.

Pulmonary hypertension to be treated in accordance with the invention is most particularly pulmonary hypertension associated with disorders of the respiratory system and/or hypoxemia, including chronic obstructive pulmonary disease, interstitial lung disease, sleep-disordered breathing, alveolar hypoventilation disorders, chronic exposure to high altitude, neonatal lung disease and alveolar-capillary dysplasia, but especially chronic obstructive pulmonary disease.

Lung fibrosis includes idiopathic pulmonary fibrosis in particular.

Compounds of the present may also be used to treat muscle diseases including muscular atrophies (e.g. disuse), muscular dystrophies (e.g. Duchenne's Muscle Dystrophy, Becker's Muscle Dystrophy, Limb-Girdle Muscle Dystrophy, Facioscapulohumeral Dystrophy), sarcopenia and cachexia.

Treatment of muscular diseases such as muscle atrophies and dystrophies is a largely unmet medical need. There are only few compounds approved for the use in assorted muscle disorders, mainly in the area of cancer-induced and HIV muscle wasting or cachexia, and a few more drugs are used off-label for these indications. In addition, most of these drugs only address the weight loss and do not specifically affect muscular growth and function. There is therefore a need for effective therapies to treat functional impairments associated with muscle diseases related to cachexia (e.g. in cancer, HIV and COPD), disuse atrophy, sarcopenia and dystrophy.

Myostatin, a member of the transforming growth factor, (TGFβ) family, is a key negative regulator of skeletal muscle mass. In double-muscle cattle and in a human body with skeletal muscle hypertrophy, different mutations in the myostatin gene were detected (McPherron et al (1997) Nature 387:83-90; Schuelke et al (2004) N. Engl. J. Med. 350:2682-2688). The important role of myostatin for skeletal muscle growth and disorders was confirmed in a wide variety of in vivo and in vitro studies. For example, muscle-specific overexpression of myostatin in mice causes loss of muscle mass (Reisz-Porszasz et al (2003) AJP— Endo. 285:876-888), whereas myostatin null mice have increased skeletal muscle mass and reduced body fat (Lin et al (2002) Biochem. Biophys. Res. Comm. 291: 701-706). In accordance systemic administration of myostatin induces cachexia (Zimmers et al (2002) Science 296:1486-1488), whereas inhibition of myostatin by, for example, the myostatin neutralizing antibody JA16 increases muscle mass and strength in wildtype and dystrophic mdx mice (Bogdanovich et al (2002) Nature 420: 418-421.2002; Wagner et al (2002)Ann. Neurol. 52: 832-836; Wolfman et al (2003) Proc. Natl. Acad. Sci. 100(26): 15842-15846). In addition, elevated myostatin levels have been observed in both experimental and clinical muscle atrophies such as in patients with Human Immunodeficiency Virus (HIV), cancer or liver cirrhosis as well as in sarcopenia of old age and under glucocorticoid-treatment (Ma et al (2003) Am. J. Physiol. Endocrinol. Metab. 285: E363-371; Gonzales-Cadavid et al (1998) Proc. Natl. Acad. Sci. 95: 14938-14943; see also Reisz-Porszasz et al (2003) AJP— Endo. 285:876-888 and Jespersen et al (2006) Scand. J. Med. Sci. Sports. 16: 74-82). These findings show the high potential of myostatin inhibitors as treatments for muscular atrophies and dystrophies.

The mode of action of myostatin is still under investigation. It is relatively well established that myostatin signals through Smad2/3 (Lee S. J. (2004) Ann. Rev. Dev. Biol. 20: 61-86). Moreover, mature myostatin has been shown to act via activin type IIb and activin receptor like kinase (ALK) receptors in adipocytes (Rebbarpragada et al (2003) Mol. Cell. Biol. 23: 7230-7242). However, respective findings in skeletal muscle cells are not described. Myostatin is believed to inhibit differentiation and cause atrophy via ALK signaling. Moreover, inhibition of ALK signaling promotes skMC differentiation and causes skMC hypertrophy.

Osteoporosis is a systemic skeletal disorder characterized by low bone mass and micro-architectural deterioration of bone tissue, with a consequent increase in bone fragility and susceptibility to fracture. The osteoporotic syndrome is multi faceted, encompassing primary disorders such as postmenopausal or age-related osteporosis, and secondary conditions that accompany disease states or medications. The mechanical properties and composition of bone matrix, along with bone mass and architecture, are critical determinants of a bone's ability to resist fracture.

Thus in a further aspect the invention includes a method for preventing or treating bone conditions which are associated with increased calcium depletion or resorption or in which stimulation of bone formation and calcium fixation in the bone is desirable in which an effective amount of an agent of the invention, or a pharmaceutically-acceptable and -cleavable ester, or acid addition salt thereof is administered to a patient in need of such treatment.

In a yet further aspect the invention includes a pharmaceutical composition for preventing or treating bone conditions which are associated with increased calcium depletion or resorption or in which stimulation of bone formation and calcium fixation in the bone is desirable comprising an agent of the invention, or a pharmaceutically-acceptable and -cleavable ester, or acid addition salt thereof, in admixture with a pharmaceutically acceptable excipient, diluent or carrier.

The compounds of the Examples herein below generally have IC₅₀ values below 1 μM. For instance, the compounds of Examples 1.1, 1.2, 1.3, 1.4, 1.6 and 1.7 have IC₅₀ values of 0.042, 0.036, 0.005, 0.150, 0.015 and 0.005 μM respectively.

The kinase activity of ALK5 is assessed by measuring radiolabelled phosphate [33P] incorporation in to the generic substrate, casein. The kinase domain of human ALK5 (amino acids 200-503) is fused to an N-terminal histidine tag. The kinase activity of ALK5 is rendered constitutive via point mutation at amino acid 204 (threonine to aspartate modification, ALK5 T204D) and the kinase construct is engineered to be expressed from a baculovirus expression construct in insect cells. The purified, recombinantly-expressed histidine-tagged ALK5 T204D protein is dissolved at 5.4 mg/ml in 50 mM Tris-HCl pH 8.0, 150 mM NaCl, 5 mM DTT. ALK5 T204D is dissolved to 2.5 μg/ml in assay buffer (Assay buffer: 20 mM Tris-HCl pH 7.4, 10 mM MgCl₂, 2 mM MnCl₂) on the day of use.

Test compounds and reference compounds are dissolved in assay buffer without DTT containing 5% (v/v) DMSO. Stock solutions of test and reference compounds are diluted in assay buffer with DTT (1.25 mM) containing 4.5% (v/v) DMSO. 10 μl of test or reference compound are added to the appropriate wells of 96 well U-bottomed plate. Total enzyme activity is determined by measuring ALK5 T204D activity in the absence of ALK5 kinase inhibitor reference compounds. Non-specific binding (NSB) is determined by measuring the activity of ALK5 T204D in the presence of ALK5 kinase inhibitor reference compounds. 10 μl of dephosphorylated casein stock solution (dephosphorylated casein is dissolved in ddH₂O at 20 mg/ml) is added per well (200 μg/well final assay concentration). 20 μl of ALK5 T204D (2.5 μg/ml solution) is added per well (50 ng/well final assay concentration). The plates are left to incubate at room temperature for 10 minutes.

10 μl of ATP mix is added to the well to initiate the reaction (0.66 nM [³³P]ATP/1 μM unlabelled ATP/well final assay concentration). The ATP mix is prepared as follows, unlabelled ATP (3 mM) is dissolved in ddH₂O and pH adjusted to 7.4. The stock concentration of [³³P]ATP is 10 μCi/μl. The appropriate volume of [³³P]ATP is added to unlabelled ATP solution such that the final assay concentration per well is 0.1 μCi. Following addition of the ATP mix, the plates are incubated at room temperature for 50 minutes. The kinase reaction is terminated by the addition of 50 μL Stop Buffer (20 mM Tris-HCl pH 7.4, 10 mM EDTA).

75 μl/well from the reaction plate is transferred to a Multiscreen-IP plate (MultiScreen-IP plates are prepared by added 50 μL of 70% (v/v) ethanol per well and incubated for 5 minutes at room temperature. The ethanol is removed by aspiration via a MultiScreen HTS Vacuum Manifold unit (Millipore, Cat no: MSVMHT500). The plates are washed twice by adding 200 μl/well ddH₂O). The MultiScreen-IP plate is incubated at room temperature for 30 minutes to allowing binding of casein to the plate. The MultiScreen-IP plates are washed three times by adding 200 μl/well 100 mM phosphoric acid solution and the gasket is carefully removed from the back of the MultiScreen-IP plate and the plate dried in the oven for 30 minutes. The MultiScreen-IP plate is backsealed, 50 μL of Microscint™20 is added, then the plates are topsealed and radiolabelled casein detected and quantified on a TopCount™ plate-reader using the ³³P scintillation protocol.

The agents of the invention are also useful as co-therapeutic agents for use in combination with other drug substances such as anti-inflammatory, bronchodilatory, antihistamine, decongestant or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with one or more other drug substances in a fixed pharmaceutical composition or it may be administered separately, before, simultaneously with or after the other drug substance(s).

Such anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 [Novartis] (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935, WO 04/26248 and WO 05/05452; LTB4 antagonists such as BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700 and WO 04/108720; LTD4 antagonists such as montelukast, pranlukast, zafirlukast, accolate, SR2640, Wy-48,252, ICI 198615, MK-571, LY-171883, Ro 24-5913 and L-648051; Dopamine receptor agonists such as cabergoline, bromocriptine, ropinirole and 4-hydroxy-7-[2-[[2-[[3-(2-phenylethoxy)-propyl]sulfonyl]ethyl]amino]ethyl]-2(3H)-benzothiazolone and pharmaceutically acceptable salts thereof (the hydrochloride being Viozano®—AstraZeneca); PDE4 inhibitors such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(TM) CC-10004 (Celgene), VM554/UM565 (Vernalis), T-440 (Tanabe), KW-4490 (Kyowa Hakko Kogyo), GRC 3886 (Oglemilast, Glenmark), WO 92/19594, WO 93/19749, WO 93/19750, WO 93/19751, WO 99/16766, WO 01/13953, WO 03/104204, WO 03/104205, WO 04/000814, WO 04/000839 and WO 04/005258 (Merck), WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607, WO 04/037805, WO 04/063197, WO 04/103998, WO 04/111044, WO 05012252, WO 05012253, WO 05/013995, WO 05/030212, WO 05/030725, WO 05/087744, WO 05/087745, WO 05/087749 and WO 05/090345 as well as those described in WO 98/18796 and WO 03/39544. A2a agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; and A2b antagonists such as those described in WO 02/42298 and WO 03/042214.

Such bronchodilatory drugs include beta-2 adrenoceptor agonists. Suitable beta-2 adrenoceptor agonists include albuterol (salbutamol), metaproterenol, terbutaline, salmeterol, fenoterol, procaterol, and especially, formoterol, carmoterol, GSK159797 and pharmaceutically acceptable salts thereof, and compounds (in free or salt or solvate form) of formula I of WO 0075114, which document is incorporated herein by reference, preferably compounds of the Examples thereof, especially a compound of formula

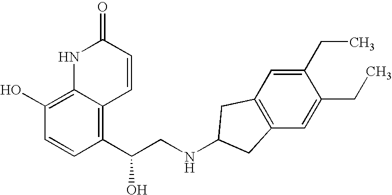

and pharmaceutically acceptable salts thereof, as well as compounds (in free or salt or solvate form) of formula I of WO 04/16601 or of formula I of WO 04/087142. Further suitable β-2-adrenoreceptor agonists include compounds, such as those described in and also compounds of EP 147719, EP 1440966, EP 1460064, EP 1477167, EP 1574501, JP 05025045, JP 2005187357, US 2002/0055651, US 2004/0242622, US 2004/0229904, US 2005/0133417, US 2005/5159448, US 2005/5159448, US 2005/171147, US 2005/182091, US 2005/182092, US 2005/209227, US 2005/256115, US 2005/277632, US 2005/272769, US 2005/239778, US 2005/215542, US 2005/215590, US 2006/19991, US 2006/58530, WO 93/18007, WO 99/64035, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/4561.8 WO. 04/46083, WO 04/80964, WO 04/087142, WO 04/89892, WO 04/108675, WO 04/108676, WO 05/33121, WO 05/40103, WO 05/44787, WO 05/58867, WO 05/65650, WO 05/66140, WO 05/70908, WO 05/74924, WO 05/77361, WO 05/90288, WO 05/92860, WO 05/92887, WO 05/90287, WO 05/95328, WO 05/102350, WO 06/56471, WO 06/74897 or WO 06/8173.

Such bronchodilatory drugs also include other anticholinergic or antimuscarinic agents, in particular ipratropium bromide, oxitropium bromide, tiotropium salts, glycopyrrolate, CHF 4226 (Chiesi) and SVT-40776, but also those described in EP 424021, U.S. Pat. No. 3,714,357, U.S. Pat. No. 5,171,744, US 2005/171147, US 2005/182091, WO 01/04118, WO 02/00652, WO 02/51841, WO 02/53564, WO 03/00840, WO 03/33495, WO 03/53966, WO 03/87094, WO 04/18422, WO 04/05285, WO 04/96800, WO 05/77361 and WO 06/48225.

Suitable dual anti-inflammatory and bronchodilatory drugs include dual beta-2 adrenoceptor agonist/muscarinic antagonists such as those disclosed in US 2004/0167167, US 2004/0242622, US 2005/182092, US 2005/256114, US 2006/35933, WO 04/74246, WO 04/74812, WO 04/89892 and WO 06/23475.

Suitable antihistamine drug substances include cetirizine hydrochloride, levocetirizine, acetaminophen, clemastine fumarate, promethazine, loratidine, desloratidine, diphenhydramine and fexofenadine hydrochloride, activastine, astemizole, azelastine, dimetinden, ebastine, epinastine, levocabastine, mizolastine and tefenadine as well as those disclosed in WO 03/099807, WO 04/026841 and JP 2004107299.

According to a further embodiment of the invention, the agents of the Invention may be employed as adjunct or adjuvant to other therapy, e.g. a therapy using a bone resorption inhibitor, for example as in osteoporosis therapy, in particular a therapy employing calcium, a ealeitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, TSE424, FC1271, Tibolone (Livial A), vitamin D or an analog thereof or PTH, a PTH fragment or a PTH derivative e.g. PTH (1-84), PTH (1-34), PTH (1-36), PTH (1-38), PTH (1-31)NH₂ or PTS 893.

In accordance with the foregoing, the present invention also provides a method for the treatment of an obstructive or inflammatory airways disease which comprises administering to a subject, particularly a human subject, in need thereof an agent of the invention, or a pharmaceutically acceptable salt or solvate thereof, as hereinbefore described. In another aspect, the invention provides an agent of the invention, or a pharmaceutically acceptable salt or solvate thereof, as hereinbefore described for use in the preparation of a medicament for the treatment of an obstructive or inflammatory airways disease.

The agents of the invention may be administered by any appropriate route, e.g. orally, for example in the form of a tablet or capsule; parenterally, for example intravenously; topically to the skin, for example in the treatment of psoriasis; intranasally, for example in the treatment of, hay fever; or, preferably, by inhalation, particularly in the treatment of obstructive or inflammatory airways diseases. In particular, the agents of the invention may be delivered as an inhalable formulation for the treatment of COPD and asthma.

In a further aspect, the invention also provides a pharmaceutical composition comprising an agent of the invention in free form or in the form of a pharmaceutically acceptable salt or solvate thereof, optionally together with a pharmaceutically acceptable diluent or carrier therefor. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the active ingredient is a nebulizable aqueous, organic or aqueous/organic dispersion, the inhalation device may be a known nebulizer, for example a conventional pneumatic nebulizer such as an airiet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers. Where the inhalable form of the active ingredient is the finely divided particulate form, the inhalation device may be, for example, a dry powder inhalation device adapted to deliver dry powder from a capsule or blister containing a dry powder comprising a dosage unit of (A) and/or (B) or a multidose dry powder inhalation (MDPI) device adapted to deliver, for example, 3-25 mg of dry powder comprising a dosage unit of (A) and/or (B) per actuation. The dry powder composition preferably contains a diluent or carrier, such as lactose, and a compound that helps to protect against product performance deterioration due to moisture e.g. magnesium stearate. Suitable such dry powder inhalation devices include devices disclosed in U.S. Pat. No. 3,991,761 (including the AEROLIZER™ device), WO 05/113042, WO 97/20589 (including the CERTIHALER™ device), WO 97/30743 (including the TWISTHALER™ device) and WO 05/37353 (including the GYROHALER™ device).

The invention also includes (A) an agent of the invention in free form, or a pharmaceutically acceptable salt or solvate thereof, in inhalable form; (B) an inhalable medicament comprising such a compound in inhalable form together with a pharmaceutically acceptable carrier in inhalable form; (C) a pharmaceutical product comprising such a compound in inhalable form in association with an inhalation device; and (D) an inhalation device containing such a compound in inhalable form.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

Quite unexpectedly, it has also been found that the compounds of formula Ia and Ib have advantageous pharmacological properties and inhibit the activity of tyrosine kinases.

It has been well-established that various receptor tyrosine kinase inhibitors are useful for the treatment of cancer, however, it is not obvious which specific compounds will be matched with which specific tyrosine kinase receptors for the treatment of which specific types of cancer. TRK receptors (NTRK genes) are correlated with the development and progression of cancer through increases in the amount of the receptors or their ligands (the neurotrophins NGF, BDNF, or NT3/4). High expression of TRK's are found in Wilm's tumor, prostate carcinoma and pancreatic cancers. High expression of TRKC is a hallmark of carcinoma. In neuroblastoma, high TRKB expression is correlated with an aggressive untreatable tumors and resistance to standard cytotoxic therapies. In mouse models of cancer metastasis, the NTRK2 gene (TRKB protein) can induce metastasis and removal of the gene reverses this metastatic potential. The bulk of evidence suggests that inhibition of TRK enzymes would block the growth and spread of various cancers where TRK is involved. Furthermore, activating mutations in TRK's are present in 7% of cancers. Thus, compounds of the invention which are TRK inhibitors are useful in the treatment of cancer, in particular the specific cancers mentioned above.

Additional research has discovered mutations in TRKB in humans that result in a partial loss of enzymatic activity of the receptor. This genetic legion results in an increase in apetite and obesity (hyperphagic obesity). Similar results have been obtained in mouse models, thus strengthening the hypothesis that lowering TRKB activity could serve to modulate feeding behavior, and would be useful in the treatment of disorders such as anorexia.

Quite unexpectedly, it has also been found that compounds of Formula Ia and Ib inhibit FLT-3 and ROS, which are also useful targets for cancer therapy with respect to acute lymphoid cancers and glioblastoma.

Several lines of evidence have implicated NTRK1 (TrkA) and its closely related family members NTRK2 (TrkB) and NTRK3 (TrkC) in the development and progression of cancer, possibly by upregulation of either the receptor, their ligand (Nerve Growth Factor, Brain Derived Neurotropic Factor, Neurotrophins) or both. Accordingly, the compounds of the invention are useful in the treatment of cancer by inhibiting the development and/or progression of the cancer.

The mechanisms by which Trk family kinase receptors promote tumorigenesis are only partially understood. It has been shown that Trk kinase receptors are able to control tumor cell growth and survival as well as differentiation, migration and metastasis. It has been recently demonstrated that NTRK2 is a potent inhibitor of anoikis (apoptosis induced by loss of attachment of a cell to its matrix). By activating the Phosphatidylinositol-3-kinase/Protein Kinase B signaling pathway, NTRK2 was shown to promote the survival of non-transformed epithelial cells in 3-dimensional cultures and to induce tumor formation and metastasis of those cells in immunocompromised mice.

Several studies suggest a role for Trk family members, especially NTRK1 and NTRK2 in pancreatic cancer: i) high expression of various members of the Trk family and their cognate ligands have been shown in tissue samples from patients with pancreatic cancer. ii) NTRK2 overexpression has recently been linked to a malignant, highly metastatic phenotype of pancreatic cancer. iii) high expression of NTRK1/NGF, has been correlated with enhanced proliferation, invasive behavior and pain in PC patients. iv) nerve growth factor has been shown to increase the invasive potential of pancreatic cancer cell lines. A recent study suggests that overexpression of TrkA in pancreatic cancer might be caused by methylation of negative regulatory AP-1 sites in the promoter region of TrkA.

Gene rearrangements involving NTRK1 are a hallmark of a subset of papillary thyroid cancers. Thyroid-specific TRK oncogenes are generated by rearrangements of the NTRK1 gene with three different activating genes, namely TPR, TPM3, and TFG.

Several loss of function mutations in thr TrkA are responsible for congenital insensitivity to pain with anhidrosis (CIPA), a disorder characterized by a lack of pain sensation and anhidrosis. More recently, an antagonistic TrkA antibody has been shown to be efficacious in inflammatory and neupathic pain animal models. In addition, TrkA and NGF have been implicated in eliciting cancer related pain. It was shown that NGF secreted by tumor cell and tumor invading macrophages secret NGF which directly stimulates TrkA located on peripheral pain fibers. Using various tumor models in both mouse and rats it was demonstrated that neutralizing NGF with a monoclonal antibody inhibits cancer related pain to a degree similar or superior to the highest tolerated dose of morphine. Therefore, a selective inhibitor of TrkA can be used in the treatment of pain associated with cancer.

Other non-oncology indications for a Trk inhibitor include atopic dermatitis and psoriasis.

Compounds of the present invention are assayed to measure their capacity to selectively inhibit cell proliferation of Ba/F3 cells expressing activated TrkA, B or C through fusion to the dimerization domain of Tel (ETV6) transcription factor as well as Ba/F3 cells co-expressing full length rTrkA and mNGF compared with parental BaF3 cells.

### Inhibition of Cellular TrkA/B/C Dependent Proliferation

Luciferase expressing Ba/F3 murine pro-B cells are transformed with Tel-TrkA/B/C or TrkA/NGF. Cells are maintained in RPMI/10% fetal calf serum (RPMI/FCS) supplemented with penicillin 50 μg/mL, streptomycin 50 μg/mL and L-glutamine 200 mM. Untransformed Ba/F3 cells are similarly maintained with the addition of murine recombinant IL3. Cells are dispensed into 384-well format plate at 5000 cell/well in 50 μL of culture medium. Compounds of the invention are dissolved and diluted in dimethylsufoxide (DMSO). Twelve point 1:3 serial dilutions are made into DMSO to create concentrations gradient ranging typically from 10 mM to 0.05 μM. Cells are added with 50 nL of diluted compounds and incubated for 48 hours in cell culture incubator. Luminiscent signal is measured following the addition of Bright Glo® (D (Promega) luciferase substrate. IC₅₀ values are calculated by linear regression analysis of the percentage inhibition of each compound at 12 concentrations.

### Upstate KinaseProfiler™—Radio-Enzymatic Filter Binding Assay

Compounds of the invention are assessed for their ability to inhibit individual members of a panel of kinases (a partial, non-limiting list of kinases includes: Abl, Aurora, cSrc, TPR-Met, Tie2, MET, FGFR3, Axl, Bmx, BTK, c-kit, CHK2, Flt3, MST2, p70S6K, PDGFR, PKB, PKCo, Raf, ROCK-II, Rsk1, SGK, TrkA, TrkB and TrkC). The compounds are tested in duplicates at a final concentration of 10 μM following this generic protocol. The kinase buffer composition and the substrates vary for the different kinases included in the “Upstate KinaseProfiler™” panel. The compounds are tested in duplicates at a final concentration of 10 μM following this generic protocol. Kinase buffer (2.5 μL, 10× —containing MnCk when required), active kinase (0.001-0.01 Units; 2.5 μL), specific or Poly(Glu4-Tyr) peptide (5-500 μM or 0.01 mg/ml) in kinase buffer and kinase buffer (50 μM; 5 μL) are mixed in an eppendorf on ice. A Mg/ATP mix (10 μL; 67.5 (or 33.75) mM MgCl₂, 450 (or 225) μM ATP and 1 μCi/μl [y-³²P]-ATP (3000 Ci/mmol)) is added and the reaction is incubated at about 30° C. for about 10 minutes. The reaction mixture is spotted (20 ΞL) onto a 2 cm×2 cm P81 (phosphocellulose, for positively charged peptide substrates) or Whatman No. 1 (for Poly (Glu-4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Example compounds 3.1 to 3.7, for example, all exhibit an IC₅₀ of less than 1 μM.

Quite unexpectedly, it has also been found that the compounds of Formula Ia and Ib have advantageous pharmacological properties and inhibit the activity of the lipid kinases, such as the PI3-kinase and/or members of the PI3-kinase-related protein kinase family (also called PIKK and include DNA-PK, ATM, ATR, hSMG-1 and mTOR), such as the DNA protein-kinase, and may be used to treat disease or disorders which depend on the activity of said kinases.

The phosphatidylinositol-3′-OH kinase (PI3K) pathway is one of the central signaling pathways that exerts its effect on numerous cellular functions including cell cycle progression, proliferation, motility, metabolism and survival. An activation of receptor tyrosine kinases causes PI3K to phosphorylate phosphatidylinositol-(4,5)-diphosphate, resulting in membrane-bound phosphatidylinositol-(3,4,5)-triphosphate. The latter promotes the transfer of a variety of protein kinases from the cytoplasm to the plasma membrane by binding of phosphatidylinositol-(3,4,5)-triphosphate to the pleckstrin-homology (PH) domain of the kinase. Kinases that are key downstream targets of PI3K include phosphoinositide-dependent kinase 1 (PDK1) and AKT (also known as Protein Kinase B). Phosphorylation of such kinases then allows for the activation or deactivation of numerous other pathways, involving mediators such as GSK3, mTOR, PRAS40, FKHD, NF-κB, BAD, Caspase-9, and the like. An important negative feedback mechanism for the PI3K pathway is PTEN, a phosphatase that catalyses the dephosphorylation of phosphatidylinositol-(3,4,5)-triphosphate to phosphorylate phosphatidylinositol-(4,5)-diphosphate. In more than 60% of all solid tumors, PTEN is mutated into an inactive form, permitting a constitutive activation of the PI3K pathway. As most cancers are solid tumors, such an observation provides evidence that a targeting of PI3k itself or individual downstream kinases in the PI3K pathway provide a promising approach to mitigate or even abolish the dysregulation in many cancers and thus restore normal cell function and behaviour. This, however, does not exclude that other mechanisms may be responsible for the beneficial effects of PI3K activity modifying agents such as those in the present invention.

Having regard to their inhibitory effect on phosphatidylinositol 3-kinase enzymes, compounds of Formula Ia and Ib in free or pharmaceutically acceptable salt form, are useful in the treatment of conditions which are mediated by the activation (including normal activity or especially overactivity) of one or more of the members of the PI3 kinase family, especially PI3 kinase enzyme, such as proliferative, inflammatory or allergic conditions, obstructive airways diseases and/or disorders commonly occurring in connection with transplantation.

“Treatment” in accordance with the invention may be therapeutic, e.g. symptomatic, and/or prophylactic. Preferred is the treatment of warm-blooded animals, especially humans.

An aspect of the present invention provides a compound of Formula Ia or Ib for use or the use thereof in the treatment of a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, lymphomas, a mammary carcinoma or a leukemia. Other diseases include Cowden syndrome, Lhermitte-Dudos disease and Bannayan-Zonana syndrome, or diseases in which the PI3K/PKB pathway is aberrantly activated.

Compounds according to the invention are also of use in the treatment of inflammatory or obstructive airways (respiratory tract) diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, e.g. mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma can be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Compounds of Formula Ia and Ib can be of use for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy.

The invention also to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, compounds of the invention are also of use in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also of use in the treatment of inflammatory or allergic conditions of the skin, for example psoriasis, contact dermatitis, atopic dermatitis, alopecia greata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, and other inflammatory or allergic conditions of the skin.

Compounds of the invention may also be used for the treatment of other diseases or conditions, such as diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minimal change nephropathy).

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, or a disorder commonly occurring in connection with transplantation.

The invention expecially relates to the use of a compound of the Formula Ia or Ib (or a pharmaceutical formulation comprising a compound of the Formula Ia or Ib) in the treatment of one or more of the diseases mentioned above and below where the disease(s) respond or responds (in a beneficial way, e.g. by partial or complete removal of one or more of its symptoms up to complete cure or remission) to an inhibition of one or more kinases of the PI3-kinase-related protein kinase family, most especially PI3 kinase (PI3K), especially where the kinase shows (in the context of other regulatory mechanisms) inadequately high or more preferably higher than normal (e.g. constitutive) activity.

Whereever the term “use” or “used” is mentioned, this is intended to include a compound of the Formula Ia or Ib for use in the prophylactic and/or therapeutic treatment of a disease of a warm-blooded animal, especially a human, preferably of one or more diseases mentioned above or below, a method of use or a method of treatment comprising administering a compound of the Formula Ia or Ib to a person in need of such treatment in an effective amount for the prophylactic and/or therapeutic treatment of a disease as mentioned above and below, the preparation or a method or preparation of a pharmaceutical formulation/preparation for use in the prophylactic and therapeutic treatment of a disease mentioned above and below, especially involving mixing a compound of the Formula Ia or Ib (as therapeutically active ingredient) with at least one pharmaceutically acceptable carrier material, including making it ready for use in such treatment (e.g. adding an instruction insert (e.g. package leaflet or the like), formulation, appropriate preparation, adaptation for specific uses, customizing and the like), and the use of a compound of the Formula Ia or Ib for such preparation, and/or all other prophylactic or therapeutic uses mentioned hereinbefore or below. All these aspects are embodiments of the present invention.

The efficacy of the compounds of Formula Ia and Ib and salts thereof as PI3 kinase inhibitors can be demonstrated as follows:

The kinase reaction is performed in a final volume of 50 μL per well of a half area COSTAR, 96 well plate. The final concentrations of ATP and phosphatidyl inositol in the assay are 5 μM and 6 μg/mL respectively. The reaction is started by the addition of PI3 kinase p110β. The components of the assay are added per well as follows:
10 μL test compound in 5% DMSO per well in columns 2-1.
Total activity is determined by addition 10 μL of 5% vol/vol DMSO in the first 4 wells of column 1 and the last 4 wells of column 12.
The background is determined by addition of 10 μM control compound to the last 4 wells of column 1 and the first 4 wells of column 12.
2 mL ‘Assay mix’ are prepared per plate:
   1.912 mL of HEPES assay buffer
   8.33 mL of 3 mM stock of ATP giving a final concentration of 5 μM per well
   1 μL of [³³P]ATP on the activity date giving 0.05 μCi per well
   30 μL of 1 mg/mL PI stock giving a final concentration of 6 μg/mL per well
   5 μL of 1 M stock MgCl₂ giving a final concentration of 1 mM per well
20 μL of the assay mix are added per well.
2 mL ‘Enzyme mix’ are prepared per plate (x μL PI3 kinase p110β in 2 mL of kinase buffer). The ‘Enzyme mix’ is kept on ice during addition to the assay plates.
20 μL ‘Enzyme mix’ are added/well to start the reaction.
The plate is then incubated at room temperature for 90 minutes.
The reaction is terminated by the addition of 50 μL WGA-SPA bead (wheat germ agglutinin-coated Scintillation Proximity Assay beads) suspension per well.
The assay plate is sealed using TopSeal-S heat seal for polystyrene microplates, PerkinElmer LAS (Deutschland) GmbH, Rodgau, Germany) and incubated at room temperature for at least 60 minutes.
The assay plate is then centrifuged at 1500 rpm for 2 minutes using the Jouan bench top centrifuge (Uouan Inc., Nantes, France).
The assay plate is counted using a Packard TopCount, each well being counted for 20 seconds.
   The volume of enzyme is dependent on the enzymatic activity of the batch in use.

Some of the compounds show a certain level of selectivity against the different paralogs PI3K alpha, beta, gamma and delta.

### Description of Biochemical Assay for DNA-PK:

The assay is conducted using the kit V7870 from Promega (SignaTECT® DNA-Dependent Protein Kinase Syste, comprises DNA-PK, biotinylated peptide substrate end further ingredients, Promega, Madison, Wis., USA), that quantitates DNA-dependent protein kinase activity, both in purified enzyme preparations and in cell nuclear extracts. DNA-PK is a nuclear serine/threonine protein kinase that requires double-stranded DNA (dsDNA) for activity. The binding of dsDNA to the enzyme results in the formation of the active enzyme and also brings the substrate closer to the enzyme, allowing the phosphorylation reaction to proceed.

DNA-PK×5 reaction buffer (250 mM HEPES, 500 mM KCl, 50 mM MgCl₂, 1 mM EGTA, 0.5 mM EDTA, 5 mM DTT, pH to 7.5 with KOH) is diluted 1/5 in deionised water and BSA (stock=10 mg/ml) is added to a final concentration of 0.1 mg/ml.

The activation buffer is made from 100 μg/ml of calf thymus DNA in control buffer (10 mM Tris-HCl (pH 7.4), 1 mM EDTA (pH 8.0)). Per tube, the reaction mix is composed of: 2.5 μl of activation or control buffers, 5 μl of ×5 reaction buffer, 2.5 μl of p53-derived biotinylated peptide substrate (stock=4 mM), 0.2 μl of BSA (stock at 10 mg/ml) and 5 μl of [y-³²P] ATP (5 μl of 0.5 mM cold ATP+0.05 μl of Redivue [y-³²P] ATP=Amersham AA0068-250 μCi, 3000 Ci/mmol, 10 μCi/μl (now GE Gealthcare Biosciences AB, Uppsala, Sweden).

The DNA-PK enzyme (Promega V5811, concentration=100 U/μL) is diluted 1/10 in ×1 reaction buffer and kept on ice until imminent use. 10.8 μl of the diluted enzyme is incubated with 1.2 μl of 100 μM compounds (diluted 1/100 in water from 10 mM stock in neat DMSO) for 10 minutes, at room temperature. During that time, 15.2 μl of the reaction mix is added to screw-capped tubes, behind Perspex glass. 9.8 μl of the enzyme is then transferred to the tubes containing the reaction mix and after 5 minutes incubation, at 30° C., the reaction is stopped by adding 12.5 μl of termination buffer (7.5 M guanidine hydrochloride).

After mixing well, a 10 μl aliquot of each tube is spotted onto a SAM2® biotin capture membrane (Promega, Madison, Wis., USA), which is left to dry for a few minutes. The membrane is then washed extensively to remove the excess free [y-³²P] ATP and nonbiotinyated proteins: once for 30 seconds in 200 ml of 2M NaCl, 3 times for 2 minutes each in 200 ml of 2M NaCl, 4 times for 2 minutes each in 2M NaCl in 1% H₃PO₄ and twice for 30 seconds each in 100 ml of deionised water. The membrane is subsequently left to air-dry at room temperature for 30-60 minutes.

Each membrane square is separated using forceps and scissors and placed into a scintillation vial, after which 8 ml of scintillation liquid (Flo-Scint 6013547 from Perkin-Elmer) is added. The amount of ³²P incorporated into the DNA-PK biotinylated peptide substrate is then determined by liquid scintillation counting.

The efficacy of the compounds of the invention in blocking the activation of the PI3K/PKB pathway can be demonstrated in cellular settings as follows:

### Protocol for the Detection of Phospho-PKB in U87MG Cells by ELISA:

U87MG cells (human glioblastoma, ATCC No. HTB-14) are trypsinized, counted in a CASY cell counter (Schärffe systems, Göttingen, Germany), diluted in fresh complete DMEM high glucose medium to load, per well, 150 μL cell suspension containing 4×10⁴ cells, and test plates incubated for 18 hours. In parallel, 50 μL of coating antibody, at the desired concentration in PBS/O is loaded in each well of the ELISA plates, and plates are kept for 2 hours at room temperature. This ELISA assays is performed in black flat-bottom 96-well plates (Microtest™, Falcon Becton-Dickinson, Ref.: 353941) sealed with Plate Sealers (Costar-Corning, Ref: 3095). Medium in plates is discarded and replaced by complete DMEM high glucose medium containing either 0.1% DMSO or 0.1% inhibitor at titers (7) between 10 mM and 0.156 mM in DMSO. After 30 minutes of contact, the medium is quickly removed by aspiration, plates are then placed on ice and immediately cells lyzed with 70 μL of Lysis buffer. In parallel, the 96 wells plates prepared with the coating antibody (1/250 diluted (in PBS/O) Anti-Akt1 C-20, goat, Santa-Cruz-1618, Santa Cruz Biotechnology, Inc., Santa Cruz, Calif., USA) are washed 3 times for 1 minute with PBS/O containing 0.05% Tween 20 and 0.1% Top-Blocks (derivative of gelatine that blocks unspecific binding sites on surfaces; Sigma-Aldrich, Fluka, Buchs, Switzerland, Ref.: 37766), and remaining protein binding sites blocked to prevent non-specific interactions with 200 μL of PBS containing 3% Top Block®, for 2 hours at room temperature. Well content is replaced with 50 μL of samples from treated cells, and plates are incubated for 3 hours at 4° C. The ELISA assays are always done in parallel with the following controls, in 6 replicates: U87MG (untreated control) or Lysis buffer alone (LB). After 3×15 minutes washes, all wells received 50 μL of the secondary antibody (1/250 diluted (in 3% top block) Anti-S473P-PKB, rabbit, Cell Signaling-9271, Cell Signaling Technologies, Inc., Danvers, Mass., USA)), and are incubated for 16 hours at 4° C. After three washes, plates are incubated with the third and conjugated antibody (1/1000 diluted (in 3% top block) anti rabbit (HRP) Jackson Immuno Research 111-035-144) for 2 hours at room temperature. Finally, the immune-complexes are washed 2 times 15 seconds with PBS/O/tween20/top block, 1 time with 200 μL of water and finally 200 μL of water are left in each test well before a 45 minute incubation in darkness. The plates are then assayed with (SuperSignal™ ELISA pico Chemiluminescent substrate, Pierce, Ref.: 27070, Pierce Biotechnology, Inc., Rockford, Ill., USA). 100 μL of substrate are added, and plates shacked for 1 minute. The luminescence is read immediately on a Top-Count NXT (Packard Bioscience) luminometer.

Example compounds 1.5, 1.8 and 1.9 are found to have IC₅₀ values of 0.106, 0.666 and 0.7531 μM respectively.

There are also experiments that can demonstrate the antitumor activity of compounds of the formula (I) in vivo.

For example, female Harlan (Indianapolis, Ind., USA) athymic nu/nu mice with s.c. transplanted human glioblastoms U87MG tumors can be used to determine the anti-tumor activity of PI3 kinase inhibitors. On day 0, with the animals under peroral Forene® (1-chloro-2,2,2-trifluoroethyldifluormethylether, Abbot, Wiesbaden, Germany) narcosis, a tumor fragment of approximately 25 mg is placed under the skin on the animals' left flank and the small incised wound is closed by means of suture clips. When tumors reach a volume of 100 mm³, the mice are divided at random into groups of 6-8 animals and treatment commences. The treatment is carried out for a 2-3 weeks period with peroral, intravenous or intra-peritoneal administration once daily (or less frequently) of a compound of formula (I) in a suitable vehicle at defined doses. The tumors are measured twice a week with a slide gauge and the volume of the tumors is calculated.

As an alternative to cell line U87MG, other cell lines may also be used in the same manner, for example,
the MDA-MB 468 breast adenocarcinoma cell line (ATCC No. HTB 132; see also In Vitro 14, 911-15 [1978]);
the MDA-MB 231 breast carcinoma cell line (ATCC No. HTB-26; see also In Vitro 12, 331 [1976]);
the MDA-MB 453 breast carcinoma cell line (ATCC No. HTB-131);
the Colo 205 colon carcinoma cell line (ATCC No. CCL 222; see also Cancer Res. 38, 1345-55 [1978]);
the DU145 prostate carcinoma cell line DU 145 (ATCC No. HTB 81; see also Cancer Res. 37, 4049-58 [1978]),
the PC-3 prostate carcinoma cell line PC-3 (especially preferred; ATCC No. CRL 1435; see also Cancer Res. 40, 524-34 [1980]) and the PC-3M prostate carcinoma cell line;
the A549 human lung adenocarcinoma (ATCC No. CCL 185; see also Int. J. Cancer 17, 62-70 [1976]),
the NCI-H596 cell line (ATCC No. HTB 178; see also Science 246, 491-4 [1989]);
the pancreatic cancer cell line SUIT-2 (see Tomioka et al., Cancer Res. 61, 7518-24 [2001]).

The compounds of the invention are also useful as inhibitors of the tyrosine kinase activity of Janus kinases, including JAK-2 and JAK-3 kinases, as well as the lipid kinase activity of phosphoinositide 3-kinase. Consequently, the compounds may be useful in the therapy of proliferative diseases such as tumor diseases, leukaemias, polycythemia vera, essential thrombocythemia, and myelofibrosis with myeloid metaplasia. Through the inhibition of JAK-3 kinase, compounds of the invention also have utility as immunosuppressive agents, for example for the treatment of diseases such as organ transplant rejection, lupus, multiple sclerosis, rheumatoid arthritis, psoriasis, dermatitis, Crohn's disease, type-I diabetes and complications from type-1 diabetes.

As mentioned above, the compounds of the invention may be administered alone or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents.

In the context of their Janus kinase inhibitory activity, a compound of Formula Ia or Ib can, besides or in addition, be administered especially for tumor therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Therapeutic agents for possible combination are especially one or more antiproliferative, cytostatic or cytotoxic compounds, for example one or several agents selected from the group which includes, but is not limited to, an inhibitor of polyamine biosynthesis, an inhibitor of a protein kinase, especially of a serine/threonine protein kinase, such as protein kinase C, or of a tyrosine protein kinase, such as the EGF receptor tyrosine kinase, e.g. Iressa®, the VEGF receptor tyrosine kinase, e.g. PTK787 or Avastin®, or the PDGF receptor tyrosine kinase, e.g. STI571 (Glivec®), a cytokine, a negative growth regulator, such as TGF-β or IFN-β, an aromatase inhibitor, e.g. letrozole (Femara®) or anastrozole, an inhibitor of the interaction of an SH2 domain with a phosphorylated protein, antiestrogens, topoisomerase I inhibitors, such as irinotecan, topoisomerase II inhibitors, microtubule active agents, e.g. paclitaxel or an epothilone, alkylating agents, antiproliferative antimetabolites, such as gemcitabine or capecitabine, platin compounds, such as carboplatin or cis-platin, bisphosphonates, e.g. AREDIA® or ZOMETA®, and monoclonal antibodies, e.g. against HER2, such as trastuzumab.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

### JAK/TYK-Kinase Family Profiling Assays

The efficacy of the compounds of the invention as inhibitors of JAK/TYK kinase activity can be demonstrated as follows:

All four kinases of the JAK/TYK-kinase family were used as purified recombinant GST-fusion proteins, containing the active kinase domains. GST-JAK1(866-1154), GST-JAK3(811-1124), and GST-TYK2(888-1187) were expressed and purified by affinity chromatography at the EPK biology unit. GST-JAK2(808-1132) was purchased from Invitrogen (Carlsbad, USA, #4288).

The kinase assays were based on the Caliper mobility shift assay using the LabChip 3000 systems. This technology is similar to capillary electrophoresis and uses charge driven separation of substrate and product in a microfluidic chip.

All kinase reactions were performed in 384 well microtiter plates in a total reaction volume of 18 μl. The assay plates were prepared with 0.1 μl per well of test compound in the appropriate test concentration, as described under the section “preparation of compound dilutions”. The reactions were started by combining 9 μl of substrate mix (consisting of peptide and ATP) with 9 μl of kinase dilution. The reactions were incubated for 60 minutes at 30° C. and stopped by adding 70 μl of stop buffer (100 mM Hepes, 5% DMSO, 0.1% Coating reagent, 10 mM EDTA, 0.015% Brij 35).

Fluorescently labeled synthetic peptides were used as substrates in all reactions. A peptide derived from the sequence of IRS-1 (IRS-1 peptide, FITC-Ahx-KKSRGDYMTMQIG-NH2) was used for JAK1 and TYK2 and a peptide named JAK3tide (FITC-GGEEEEYFELVKKKK-NH2) for JAK2 and JAK3. Specific assay conditions are described in Table 1:

TABLE 1
Assay conditions of individual kinase assays

| Kinase | JAK1 | JAK2 | JAK3 | TYK2 |
|---|---|---|---|---|
| Buffer | 50 mM Hepes | 50 mM Hepes | 50 mM Hepes | 50 mM Hepes |
| | pH 7.5, | pH 7.5, | pH 7.5, | pH 7.5, |
| | 0.02% Tween | 0.02% Tween | 0.02% Tween | 0.02% Tween |
| | 20, 1 mM DTT, | 20, 1 mM DTT, | 20, 1 mM DTT, | 20, 1 mM DTT, |
| | 0.02% BSA, | 0.02% BSA, | 0.02% BSA, | 0.02% BSA, |
| | 12 mM MgCl2 | 9 mM MgCl2 | 1.5 mM MgCl₂ | 9 mM MgCl2 |
| DMSO | 0.6% | 0.6% | 0.6% | 0.6% |
| Kinase conc. | 50 nM | 1.8 nM | 6 nM | 40 nM |
| Substrate peptide | 5 μM | 2 μM | 2 μM | 5 μM |
| conc. | | | | |
| ATP conc. | 40 μM | 20 μM | 80 μM | 30 μM |

The terminated reactions were transferred to the Caliper LabChip 3000 reader and the turnover of each reaction was measured by determining the substrate/product ratio.

Example compounds 2.1, 2.2, 2.3, 2.4, 2.5, 2.6 and 2.7 are found to have JAK2 IC₅₀ values of 0.016, 0.008, 0.014, 0.020, 0.021, 0.011 and 0.013 μM respectively.

### Preparation of Compound Dilutions

Test compounds were dissolved in DMSO (10 mM) and transferred into 1.4 mL flat bottom or V-shaped Matrix tubes carrying a unique 2D matrix chip by individual compound hubs. The numbers of these chips were distinctively linked to the individual compound identification numbers. The stock solutions were stored at −20° C. if not used immediately. For the test procedure the vials were defrosted and identified by a scanner whereby a working sheet was generated that guided the subsequent working steps.

Compound dilutions were made in 96 well plates. This format enabled the assay of maximally 40 individual test compounds at 8 concentrations (single points) including 4 reference compounds. The dilution protocol included the production of pre-dilution plates, master plates and assay plates:

Pre-dilution plates: 96 polypropylene well plates were used as pre-dilution plates. A total of 4 pre-dilution plates were prepared including 10 test compounds each on the plate positions A1-A10, one standard compound at A11 and one DMSO control at A12. All dilution steps were done on a HamiltonSTAR robot.
Master plates: 100 μL of individual compound dilutions including standard compound and controls of the 4 “pre-dilution plates” were transferred into a 384 “master plate” including the following concentrations 1′820, 564, 182, 54.6, 18.2, 5.46, 1.82 and 0.546 μM, respectively in 90% of DMSO.
Assay plates: Identical assay plates were then prepared by pipetting 100 mL each of compound dilutions of the master plates into 384-well “assay plates”. In the following the compounds were mixed with 9 μL of assays components plus 9 μL enzyme corresponding to a 1:181 dilution steps enabling the final concentration of 10, 3.0, 1.0, 0.3, 0.1, 0.03, 0.01 and 0.003 μM, respectively. The preparation of the master plates were handled by the Matrix PlateMate Plus robot and replication of assay plates by the HummingBird robot.

On the basis of these studies, a compound of the invention shows therapeutic efficacy especially against disorders dependent on protein kinase, especially proliferative diseases mediated by JAK/TYK kinase activity.

The invention is illustrated by the following Examples.

Abbrevations used in the Examples have the following meanings:
ACOH acetic acid
DCM dichloromethane
DIPEA N,N-diisopropylethylamine
DME 1,2-dimethoxyethane
DMF N,N-dimethylformamide
DMSO dimethylsulfoxide
Et₃N triethylamine
Et₂O diethylether
EtOAc ethyl acetate
EtOH ethanol
h hour
HATU O-(7-azabenzotriazol-1-yl)-N,N,N′,N′-tetramethyluronium hexafluoro-phosphate
HCl hydrochloric acid
HPLC High Performance Liquid Chromatography
MeOH methanol
min minute(s)
ml millilitre(s)
MS mass spectroscopy
MS-ES electrospray mass spectrometry
MW microwave
NaBH(OAc)₃ sodium triacetoxyborohydride
Na₂CO₃ sodium carbonate
N₂H₄ hydrazine
NaHCO₃ sodium hydrogencarbonate
NaN₃ sodium azide
NaOH sodium hydroxyde
Na₂SO₄ sodium sulfate
NBS N-bromosuccinimide
NH₃ ammonia
NMM N-methylmorpholine
NMP 1-methyl-2-pyrrolidone
NMR nuclear magnetic resonance
PdCl₂(PPh₃)₂ dichlorobis(triphenylphosphine)-palladium (II)
Pd(PPh₃)₄ tetrakis(triphenylphosphine)palladium
PPh₃ triphenylphosphine
prep-HPLC preparative high pressure liquid chromatography; Waters system. Column: reversed phase SunFire™ Prep (100×30 mm), C18 OBD, 5 μM. Gradient elution (CH₃CN/water with 0.1% TFA), generally product obtained as a TFA salt after lyophilization.
R_{F} retention factor
R_{f} ratio of fronts in TLC
RT room temperature
SCX strong cation exchange
SiO₂ silica
t_{R} retention time
TBME tert-butyl-methylether
TFA trifluoroacetic acid
Ti(OiPr)₄ titanium (IV) isopropoxyde
TLC thin layer chromatography
UV ultraviolet
W watt

### EXAMPLES

Examples of the present invention include compounds of formula IIb

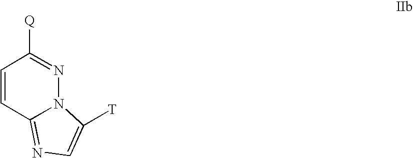

where Q and T are as shown in Tables 1, 2 and 3 below. The method of preparation being described hereinafter.

TABLE 1

| | | | [M + H]⁺ |
|---|---|---|---|
| | | | or |
| Ex. | T | Q | [M − H]⁻ |
| 1.1 | 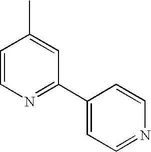 | 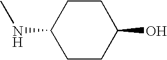 | 387 |
| 1.2 | 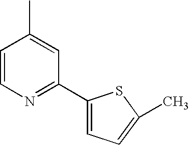 | 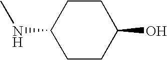 | 406 |
| 1.3 | 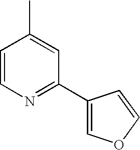 | 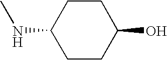 | 376 |
| 1.4 | 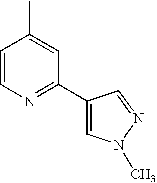 | 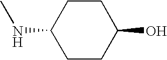 | 390 |
| 1.5 | 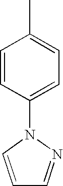 | 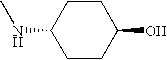 | 375 |
| 1.6 | 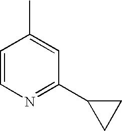 | 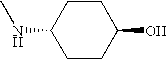 | 350 |
| 1.7 | 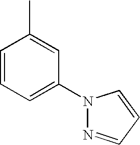 | 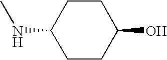 | 375 |
| 1.8 | 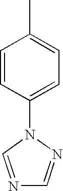 | 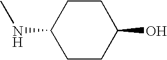 | 376 |
| 1.9 | 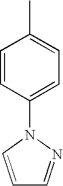 | 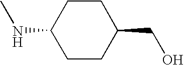 | 389 |

TABLE 2

| Ex | T | Q | [M + H]⁺ |
|---|---|---|---|
| 2.1 | 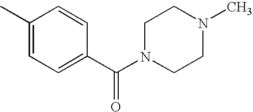 | 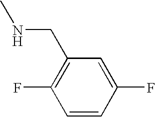 | 463 |
| 2.2 | 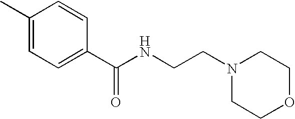 | 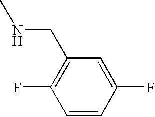 | 493 |
| 2.3 | 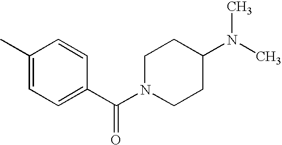 | 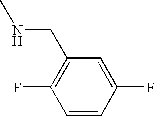 | 491 |
| 2.4 | 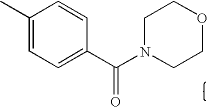 | 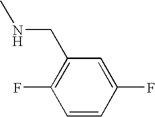 | 450 |
| 2.5 | 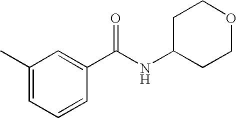 | 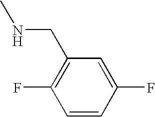 | 464 |
| 2.6 | 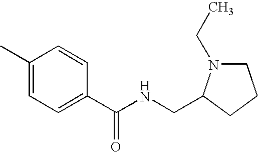 | 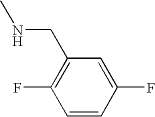 | 491 |
| 2.7 | 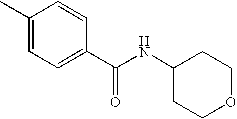 | 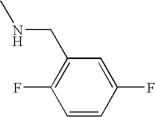 | 464 |

TABLE 3

| | | | [M + H]⁺ or |
|---|---|---|---|
| Ex. | Q | T | [M − H]⁻ |
| 3.1 | 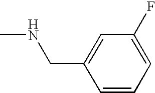 | 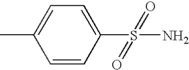 | 398 |
| 3.2 | 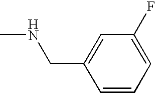 | 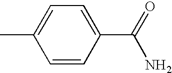 | 362 |
| 3.3 | 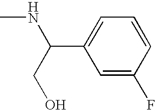 | 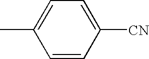 | 374 |
| 3.4 | 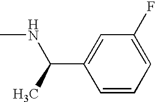 | 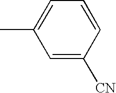 | 358 |
| 3.5 | 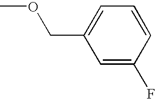 | 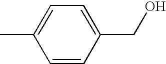 | 350 |

LCMS are recorded on an Agilent 1100 LC system with a Waters Xterra MS C18 4.6×100 5 μM column, eluting with either 5-95% 10 mM aqueous ammonium bicarbonate in acetonitrile over 2.5 minutes, with negative ion electrospray ionization or 5-95% water+0.1% TFA in acetonitrile with positive ion electrospray. Mass spectra can also be obtained under positive/negative ion electrospray ionisation conditions with LC gradient elution of 5% to 95% acetonitrile-water in the presence of 0.1% formic acid. [M+H]⁺ and [M−H]⁻ refer to mono-isotopic molecular weights. The Biotage Optimizer™ microwave synthesizer and the EmryOptimizer microwave oven are used in the standard configuration as delivered.

### Preparation of Intermediates and Final Compounds

### Example 1.1

### 4-(3-[2,4′]Bipyridinyl-4-yl-imidazo[1,2-b]pyridazin-6-ylamino)-cyclohexanol

### Step 1: 6-Chloro-imidazo[1,2-b]pyridazine

To a solution of bromoacetaldehyde (2 eq, 101 mmol, 12 g) in dimethoxyethane (200 ml) is added 3-amino-6-chloro-pyridazine (1 eq, 51 mmol, 7 g) at room temperature. The reaction is left to stir for 24 hours. The crude product is collected by filtration and dissolved in water (15 ml). The aqueous solution is then treated with sodium bicarbonate to pH=8 and cooled overnight before collecting the product, 6-Chloro-imidazo[1,2-b]pyridazine, by filtration ¹H nmr (MeOD) 8.15 (1H, s), 8.05 (1H, d, J=9.58 Hz), 7.80 (1H, s) and 7.32 (1H, d, J=9.58 Hz).

### Step 2: 3-Bromo-6-chloro-imidazo[1,2-b]pyridazine

To 6-chloro-imidazo[1,2-b]pyridazine (1 eq, 17 mmol, 2.4 g) in acetic acid (10 ml) under inert atmosphere, is added dropwise bromine (1 eq, 17 mmol, 0.82 ml). After 4 hours stirring at room temperature, the reaction mixture is filtered and dried under vacuum to give 3-bromo-6-chloro-imidazo[1,2-b]pyridazine ¹H nmr (MeOD) 8.42 (1H, d, J=9.81 Hz), 8.07 (1H, s) and 7.91 (1H, d, J=9.48 Hz).

### Step 3: 4-(3-Bromo-imidazo[1,2-b]pyridazin-6-ylamino)-cyclohexanol

To a solution of trans-4-aminocyclohexanol (5 eq, 2.5 g, 21.5 mmol) and NaHCO₃ (1 eq, 361 mg, 4.3 mmol) in N-methyl-2-pyrrolidone (NMP) (2 ml) is added 3-bromo-6-chloro-imidazo[1,2-b]pyridazine (1 eq, 1.0 g, 4.3 mmol). The reaction is heated in a microwave at 180° C. for 40 minutes. The mixture is diluted with water (20 ml) and extracted with EtOAc. The combined organic portions are washed with brine, then dried (MgSO₄) and concentrated in vacuo. Purification by flash chromatography (10% EtOAc/MeOH) gives 4-(3-bromo-imidazo[1,2-b]pyridazin-6-ylamino)-cyclohexanol.

### Step 4: 4-[3-(2-Chloro-pyridyl)-imidazo[1,2-b]pyridazin-6-ylamino]-cycloxhexanol

To a solution of 4-(3-bromo-imidazo[1,2-b]pyridazine-6-ylamino)cyclohexanol (1 eq, 8.7 mmol, 2.7 g), 3-chloropyrid-4-yl boronic acid (1.5 eq, 13 mmol, 2.05 g), Na₂CO₃ (2 eq, 17.4 mmol, 1.84 g) in dioxane (6.0 ml) and water (3 ml), under inert atmosphere is added bis(triphenylphosphine)palladium II chloride (0.1 eq, 0.87 mmol, 609 mg). The reaction mixture is heated in a microwave at 80° C. for 2 hours. The mixture is diluted with H₂O (50 ml) and extracted with EtOAc. The combined organic portions are washed with brine, then dried (MgSO₄) and concentrated in vacuo. The residue is purified by silica chromatography eluting with 2-10% EtOAc in MeOH to afford the desired final compound, 4-[3-(2-chloro-pyridyl)-imidazo[1,2-b]pyridazin-6-ylamino]-cycloxhexanol; [M+H]⁺ 345, 347.

### Step 5: 4-(3-[2,4′]Bipyridinyl-4-yl-imidazo[1,2-b]pyridazin-6-ylamino)-cyclohexanol

To 4-[3-(2-chloro-pyridin-4-yl)-imidazo[1,2-b]-pyridazin-6-ylamino]-cyclohexanol (1 eq, 100 mg, 0.29 mmol) 4-pyridyl boronic acid (1.5 eq, 0.43 mmol, 54 mg), Na₂CO₃ (2 eq, 0.58 mmol, 62 mg) in dioxane (1 ml) and H₂O (0.33 ml), under inert atmosphere is added Bis(triphenylphosphine)palladium II chloride (0.1 eq, 0.029 mmol, 21 mg). The reaction is heated in a microwave at 80° C. for 2 hours. The mixture is diluted with H₂O (5 ml) and extracted with EtOAc. The combined organic portions are washed with brine, then dried (MgSO₄) and concentrated in vacuo. The residue is purified by silica chromatography eluting with 20% EtOAc in MeOH to afford the final compound, 4-(3-[2,4′]Bipyridinyl-4-yl-imidazo[1,2-b]pyridazin-6-ylamino)-cyclohexanol; [M+H]⁺ 387.

### Examples 1.2 to 1.4

These compounds, namely
4-{3-[2-(5-Methyl-thiophen-2-yl)-pyridin-4-yl]-imidazo[1,2-b]pyridazin-6-ylamino}-cyclohexanol (Ex. 1.2),
4-[3-(2-Furan-3-yl-pyridin-4-yl)-imidazo[1,2-b]pyridazin-6-ylamino]-cyclohexanol (Ex. 1.3) and
4-{3-[2-(1-Methyl-1H-pyrazol-4-yl)-pyridin-4-yl]-imidazo[1,2-b]pyridazin-6-ylamino}-cyclohexanol (Ex. 1.4)
   are prepared using procedures that are analogous to those used to prepare the compounds of Example 1.1.

### Example 1.5

### 4-[3-(4-Pyrazol-1-yl-phenyl)-imidazo[1,2-b]pyridazin-6-ylamino]-cyclohexanol

4-(3-Bromo-imidazo[1,2-b]pyridazin-6-ylamino)-cyclohexanol (150 mg; 0.463 mmol) (Ex. 1.1 Step 3) is dissolved in DMF (3 ml) and treated at RT with [4-(1H-pyrazol-1-yl)phenyl]boronic acid (137 mg; 0.649 mmol), potassium carbonate (1M soln. in H₂O; 2.1 ml) and bis(triphenylphosphine) palladium(II)dichloride (16.6 mg; 0.023 mmol) under an atmosphere of argon. The dark yellow reaction mixture is stirred at 120° C. for 20 min at 300 W in an EmryOptimizer microwave oven. The dark brown suspension is freed from solvent under reduced pressure and purified by chromatography (40 g Redisep, ISCO Sg-100; eluting with CH₂Cl₂/CH₃OH 95:5), followed by recrystallization from EtOAc, to obtain the title compound as white crystals; [M+H]⁺ 375.

### Examples 1.6 to 1.7

These examples namely,
4-[3-(2-Cyclopropyl-pyridin-4yl)-imidazo[1,2-b]pyridazin-6-ylamino]-cyclohexanol (Ex. 1.6) and
4-[3-(3-Pyrazol-1-yl-phenyl)-imidazo[1,2-b]pyridazin-6-ylamino]-cyclohexanol (Ex. 1.7) are prepared using procedures that are analogous to those used to prepare the compounds of Example 1.5.

### Example 1.8

### 4-[3-(4-[1,2,4]Triazol-1-yl-phenyl)-imidazo[1,2-b]pyridazin-6-ylamino]-cyclohexanol

### Step 1: 4-[3-(4-Fluoro-phenyl)-imidazo[1,2-b]pyridazin-6-ylamino]-cyclohexanol

This compound is prepared analogously to Ex. 1.5 by replacing [4-(1H-pyrazol-1-yl)phenyl]boronic acid with 4-fluoro-boronic acid. [M+H]⁺ 327.

### Step 2: 4-[3-(4-[1,2,4]Triazol-1-yl-phenyl)-imidazo[1,2-b]pyridazin-6-ylamino]-cyclohexanol

4-[3-(4-Fluoro-phenyl)-imidazo[1,2-b]pyridazin-6-ylamino]-cyclohexanol (65.3 mg; 0.2 mmol) is dissolved in DMF (5 ml) and treated with 1H-[1,2,4]triazole (28 mg; 0.2 mmol) and potassium carbonate (56 mg; 0.2 mmol). The mixture is heated to 220° C. in an EmryOptimizer microwave oven (300 W). After cooling to RT, EtOAc (50 ml) is added and the organics are washed with water twice. The organic layer is freed from solvent under reduced pressure. Purification is done by flash chromatography (silica gel [0.040-0.063 mm] Merck 1.09.385.1000]; eluting with 94:6 CH₂Cl₂/CH₃OH), followed by lyophilisation from dioxan, to obtain the title compound as off-white powder; [M+H]⁺ 376.

### Example 1.9

### {4-[3-(4-Pyrazol-1-yl-phenyl)-imidazo[1,2-b]pyridazin-6-ylamino]-cyclohexyl}-methanol

### Step 1: [4-(3-Bromo-imidazo[1,2-b]pyridazin-6-ylamino)-cyclohexyl]-methanol

This compound is prepared analogously to 4-(3-bromo-imidazo[1,2-b]pyridazin-6-ylamino)-cyclohexanol (Ex. 1.1 Step 3) by replacing trans-4-aminocyclohexanol with (4-amino-cyclohexyl)-methanol instead; [M+H]⁺ 327.

### Step 2: 4-[3-(4-Pyrazol-1-yl-phenyl)-imidazo[1,2-b]pyridazin-6-ylamino]-cyclohexyl)-methanol

The title compound is prepared analogously to Ex. 1.5 by replacing 4-(3-bromo-imidazo[1,2-b]pyridazin-6-ylamino)-cyclohexanol (Ex. 1.1 Step 3) with [4-(3-bromo-imidazo[1,2-b]pyridazin-6-ylamino)-cyclohexyl]-methanol (Ex. 1.9 step 1); [M+H]⁺ 389.

### Example 2.1

### {4-[6-(2,5-Difluoro-benzylamino)-imidazo[1,2-b]pyridazin-3-yl]-phenyl}-(4-methyl-piperazin-1-yl)-methanone

### Step 1: (3-Bromo-imidazo[1,2-b]pyridazin-6-yl)-(2,5-difluoro-benzyl)-amine

To a suspension of 3-bromo-6-chloro-imidazo[1,2-b]pyridazine (1.00 g, 4.30 mmol) [example 1.1 step 2] and 2,5-difluorobenzylamine (1.03 ml, 8.60 mmol) is added KF (2.50 g, 43.0 mmol) at RT. The reaction mixture is heated to 180° C. for 1 h. After cooling to RT, the reaction mixture is diluted with EtOAc and washed with saturated aqueous Na₂CO₃ solution (3×) and saturated aqueous NaCl solution (1×). The organic layer is dried (Na₂SO₄), filtered, and concentared under reduced pressure. The resulting solid is triturated with EtOAc to afford the title compound as an off-white solid. MS-ES: [M+H]⁺ 341.

### Step 2: 4-[6-(2,5-Difluoro-benzylamino)-imidazo[1,2-b]pyridazin-3-yl]-benzoic acid

To a suspension of (3-bromo-imidazo[1,2-b]pyridazin-6-yl)-(2,5-difluoro-benzyl)-amine (400 mg, 1.14 mmol), 4-carboxyphenylboronic acid (240 mg, 1.37 mmol), and K₂CO₃ (2.00 ml, 4.00 mmol, 2 M in H₂O) in DME (5 ml) is added PdCl₂(PPh₃)₂ (41 mg, 0.06 mmol) under argon atmosphere at RT. The reaction mixture is heated to 150° C. for 20 min in a microwave oven. After cooling to RT, the reaction mixture is filtered through a Florisil pad. The pad is washed with EtOAc and the filtrate is discarded. Then, the pad is washed with MeOH and the filtrate is concentrated under reduced pressure to yield the crude title compound (70% purity) as an off-white solid which is used in the next step without further purification. MS-ES: [M+H]⁺ 381.

### Step 3: {4-[6-(2,5-Difluoro-benzylamino)-imidazo[1,2-b]pyridazin-3-yl]-phenyl}-(4-methyl-piperazin-1-yl)-methanone

To a solution of 4-[6-(2,5-difluoro-benzylamino)-imidazo[1,2-b]pyridazin-3-yl]-benzoic acid (54 mg, 0.100 mmol, 70% purity) in DMF (2 ml) is added HATU (50 mg, 0.130 mmol) and NMM (28 μl, 0.250 mmol) at RT. After stirring for 5 min, 1-methyl piperazine (13 μl, 0.110 mmol) is added and the mixture is stirred for another 2 h. The reaction mixture is diluted with EtOAc and washed with saturated aqueous NaHCO₃ solution (2×) and saturated aqueous NaCl solution (1×). The organic layer is dried (Na₂SO₄), filtered, and concentared under reduced pressure. The residue is purified by reverse phase prep-HPLC (Waters) to afford the title compound (Example 1) as a white solid (TFA salt). MS-ES: [M+H]⁺ 463.

### Examples 2.2 to 2.7

These examples namely,
4-[6(2,5-Difluorobenzylamino)-imidazo[1,2-b]pyridazin-3-yl]-N-(2-morpholin-4-yl-ethyl)benzamide (Ex. 2.2),
{4-[6-(2,5-Difluorobenzylamino)-imidazo[1,2-b]pyridazin-3-yl]-phenyl}-(4-dimethylamino-piperidin-1-yl)-methanone (Ex. 2.3),
{4-[6-(2,5-Difluoro-benzylamino)-imidazo[1,2-b]pyridazin-3-yl]-phenyl}-morpholin-4-yl-methanone (Ex. 2.4),
{3-[6-(2,5-Difluoro-benzylamino)-imidazo[1,2-b]pyridazin-3-yl]-N-(tetrahydro-pyran-4-yl)-benzamide (Ex. 2.5),
4-[6-(2,5-Difluoro-benzylamino)-imidazo[1,2-b]pyridazin-3-yl]-N-(1-ethyl-pyrrolidin-2-ylmethyl)-benzamide (Ex. 2.6) and
4-[6-(2,5-Difluorobenzylamino)-imidazo[1,2-b]pyridazin-3-yl]-N-(tetrahydro-pyran-4-yl)-benzamide (Ex. 2.7)
   are obtained analogously to Example 2.1 using the appropriate carboxyphenylboronic acids in Step 2 and the appropriate amines in Step 3.

### Example 3.1

### 4-[6-(3-Fluoro-benzylamino)-imidazo[1,2-b]pyridazin-3-yl]-benzenesulfonamide

### Step 1: (3-Bromo-imidazo[1,2-b]pyridazin-6-yl)-(3-fluoro-benzyl)-amine

In a sealed tube, a mixture of 3-bromo-6-chloro-imidazo[1,2-b]pyridazine (3.7 g, 15.9 mmol) [Example 1.1 step B] and 3-fluorobenzylamine (4.54 ml, 39.8 mmol) in NMP (16.5 ml) is heated at 180° C. and stirred for 3 h. The reaction mixture is cooled to RT, poured into water (300 ml) and extracted with EtOAc. The combined organic fractions are dried over Na₂SO₄, filtered and evaporated to dryness. The remaining residue is purified by Combi-Flash Companion™ (Isco Inc.) column chromatography (SiO₂; gradient elution, DCM/[DCM/MeOH—NH₃ 9:1] 95:5→3:7) to yield the title compound as a white solid. MS-ES [M+H]⁺-=321.0.

### Step 2: 4-[6-(3-Fluoro-benzylamino)-imidazo[1,2-b]pyridazin-3-yl]-benzenesulfonamide

In a sealed tube, a mixture of (3-bromo-imidazo[1,2-b]pyridazin-6-yl)-(3-fluoro-benzyl)-amine (50 mg, 0.156 mmol), 4-(4,4,5,5-tetramethyl-[1,3,2]dioxaborolan-2-yl)-benzenesulfonamide (52.9 mg, 0.187 mmol), PdCl₂(PPh₃)₂ (5.5 mg, 0.008 mmol) and a 2M Na₂CO₃ aqueous solution (0.27 ml) in DME (1 ml) is heated at 150° C. for 17 min in a microwave oven. The reaction mixture is cooled to RT, filtered and the filter cake is washed with DCM. The filtrate is evaporated to dryness and the remaining residue is purified by reverse phase prep-HPLC (Waters system) to give the title compound as a white powder. MS-ES [M+H]⁺=398.

### Examples 3.2 to 3.5

These examples namely,
4-[6(3-Fluoro-benzylamino)-imidazo[1,2-b]pyridazin-3-yl]-benzamide (Ex. 3.2)
4-{6-[(R or S)-1-(3-Fluoro-phenyl)-2-hydroxy-ethylamino]-imidazo[1,2-b]pyridazin-3-yl}-benzonitrile (Ex. 3.3),
3-{6-[(R)-1-(3-Fluoro-phenyl)-ethylamino]-imidazo[1,2-b]pyridazin-3-yl}-benzonitrile (Ex. 3.4),
{4-[6-(3-Fluoro-benzyloxy)-imidazo[1,2-b]pyridazin-3-yl]-phenyl}-methanol (Ex. 3.5),
   are obtained analogously to Example 3.1 using the appropriate benzylic amines or benzylic alcohols in Step 1 and appropriate boronic acids or esters in Step 2.

### Example 3.6 (not in the above Tables)

### (3-Fluoro-benzyl)-{3-[4-(2-methyl-2H-tetrazol-5-yl)-phenyl]-imidazo[1,2-b]pyridazin-6-yl}-amine

### Step 1: 4-[6-(3-Fluoro-benzylamino)-imidazo[1,2-b]pyridazin-3-yl]-benzonitrile

In a sealed tube, a mixture of (3-bromo-imidazo[1,2-b]pyridazin-6-yl)-(3-fluoro-benzyl)-amine (300 mg, 0.934 mmol) [example 3.1 step A], 4-cyanophenylboronic acid (165 mg, 1.12 mmol), PdCl₂(PPh₃)₂ (32.8 mg, 0.047 mmol) and a 2M Na₂CO₃ aqueous solution (1.6 ml) in DME (10 ml) is heated at 150° C. for 30 min in a microwave oven. The reaction mixture is cooled to RT, diluted with AcOEt (100 ml) and washed with water (30 ml) and brine (30 ml). The organic fraction is dried over Na₂SO₄, filtered and evaporated to dryness. The remaining residue is purified by Combi-Flash Companion™ (Isco Inc.) column chromatography (SiO₂; gradient elution, DCM/[DCM/MeOH 1:1] 98:2→9:1) to yield the title compound (257 mg, 0.748 mmol, 80%) as a white solid. MS-ES [M+1]⁺=344.

### Step 2: (3-Fluoro-benzyl)-{3-[4-(2H-tetrazol-5-yl)-phenyl]-imidazo[1,2-b]pyridazin-6-yl}-amine

In a sealed tube, a mixture of 4-[6-(3-fluoro-benzylamino)-imidazo[1,2-b]pyridazin-3-yl]-benzonitrile (257 mg, 0.748 mmol), NH₄Cl (134 mg, 2.25 mmol) and NaN₃ (146 mg, 2.25 mmol) in DMF (4 ml) is heated at 100° C. and stirred for 24 h. The reaction mixture is cooled to RT, diluted in DCM and filtered. The filtrate is concentrated to dryness and the remaining residue is triturated in MeOH. The resulting solid is collected by filtration washed with Et₂O and dried under vacuum to give the crude title compound as a beige solid. MS-ES [M+H]⁺=387.

### Step 3: (3-Fluoro-benzyl)-{3-[4-(2-methyl-2H-tetrazol-5-yl)-phenyl]-imidazo[1,2-b]pyridazin-6-yl}-amine

In a sealed tube, a mixture of (3-fluoro-benzyl)-(3-[4-(2H-tetrazol-5-yl)-phenyl]-imidazo[1,2-b]pyridazin-6-yl)-amine (70 mg, 0.18 mmol), Cs₂CO₃ (89.4 mg, 0.27 mmol) and methyliodide (0.028 ml, 0.45 mmol) in DMF (1 ml) is heated at 50° C. and stirred for 2 h. The reaction mixture is cooled to RT, diluted in EtOAc and washed with water. The organic layer is dried over Na₂SO₄, filtered, and concentrated to dryness. The remaining residue is purified by reverse phase prep-HPLC (Waters system) to give the title compound as a white powder. MS-ES [M+H]⁺=401.

### Example 3.7 (not in the above Tables)

### Tetrahydro-pyran-4-carboxylic acid {3-[6(2,5-difluoro-benzylamino)-imidazo[1,2-b]pyridazin-3-yl]-phenyl}-amide

### Step A: [3-(3-Amino-phenyl)-imidazo[1,2-b]pyridazin-6-yl]-(2,5-difluoro-benzyl)-amine

In a sealed tube, a mixture of (3-bromo-imidazo[1,2-b]pyridazin-6-yl)-(2,5-difluoro-benzyl)-amine (433 mg, 1.28 mmol) [Example 2.1 step 1], 3-aminophenylboronic acid (210 mg, 1.53 mmol), Pd(PPh₃)₄ (73.7 mg, 0.064 mmol) and a 2M Na₂CO₃ aqueous solution (2.2 ml) in DME (8 ml) is heated at 150° C. for 17 min in a microwave oven. The reaction mixture is cooled to RT, diluted with EtOAc (100 ml) and washed with a 2M Na₂CO₃ aqueous solution and brine. The organic layer is dried over Na₂SO₄, filtered and evaporated to dryness. The remaining residue is purified by Combi-Flash Companion™ (Isco Inc.) column chromatography (SiO₂; gradient elution, DCM/[DCM/MeOH—NH₃ 9:1] 95:5→7:3) to yield the title compound as an orange solid. MS-ES [M+H]⁺=352.

### Step B: Tetrahydro-pyran-4-carboxylic acid {3-[6-(2,5-difluoro-benzylamino)-im idazo[1,2-b]pyridazin-3-yl]-phenyl}-amide

To a solution of [3-(3-amino-phenyl)-imidazo[1,2-b]pyridazin-6-yl]-(2,5-difluoro-benzyl)-amine (50 mg, 0.14 mmol) and tetrahydro-pyran-4-carboxylic acid (22 mg, 0.17 mmol) in DMF (0.5 ml) are successively added NMM (0.078 ml, 0.71 mmol) and HATU (82 mg, 0.21 mmol) at RT. The reaction mixture is stirred at RT for 3 h, then directly subjected to purification by reverse phase prep-HPLC (Waters system) to give the title compound as a white powder. MS-ES [M+H]⁺=464.

1. A compound of Formula Ia or Ib 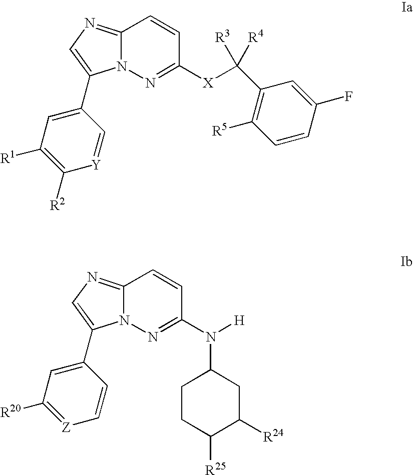
in free or salt or solvate form, wherein:
X is O or NH;
Y is CR¹³ or N;
R¹ is selected from H, CN, halo, —C(O)NR⁷R⁸ and 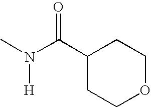
R² is selected from H, CN, morpholino, tetrazole optionally substituted by C₁-C₃ alkyl, —S(O)₂NH₂, —C(O)NR⁷R⁸ and CH₂OH,
provided that R¹ and R² are not both H and provided that when R² is other than H, R¹ is H or halo; and when R¹ is other than H, R² is H; or R¹ and R² together with the carbon atoms to which they are attached form a 6-membered heterocyclic ring containing at least one heteroatom selected from N, O and S, the heterocyclic ring being optionally substituted by C₁-C₃ alkyl or an oxo group;
R³ is selected from H, Me and CH₂OH;
R⁴ is H or C₁-C₃ alkyl;
R⁵ is H or halogen;
R⁷ is H or C₁-C₃ alkyl;
R⁸ is independently selected from H, C₁-C₆ alkyl, (CH₂)ₘhet and (CH₂)ₙNR⁹R¹⁰; or
R⁷ and R⁸ together with the nitrogen atom to which they are attached form a 5- or 6-membered heterocyclic ring optionally containing a further heteroatom selected from N, O and S, the ring being optionally substituted by C₁-C₃ alkyl or NR¹¹R¹²;
R⁹, R¹⁰, R¹¹ and R¹² are each independently selected from H and C₁-C₃ alkyl;
R¹³ is H or halo;
m and n are each independently 0, 1 or 2;
het is a 5- or 6-membered heterocyclic ring containing one or two heteroatoms selected from N, O and S, the ring being optionally substituted by C₁-C₃ alkyl;
Z is N or CR²⁶;
R²⁰ is selected from H, cyclopropyl and R²¹, provided that when Z is N, R²⁰ is other than H;
R²¹ is selected from 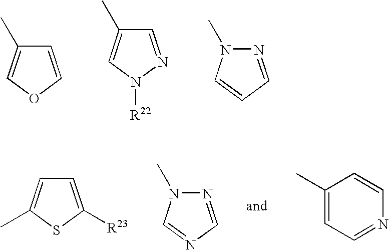
R²² and R²³ are each independently selected from H and C₁-C₃ alkyl;
R²⁴ is selected from H and OH;
R²⁵ is selected from H, OH and CH₂OH; provided that when R²⁴ is H, R²⁵ is OH or CH₂OH; and when R²⁴ is OH, R²⁵ is H; and
R²⁶ is selected from H and R²¹, provided that when R²⁰ is other than H, R²⁶ is H; and when R²⁰ is H, R²⁶ is R²¹.

2. A compound according to claim 1, wherein R¹ is selected from H, CN, halo, —C(O)NR⁷R⁸ and 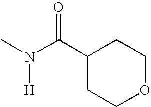
R² is selected from H, CN, morpholino, tetrazole optionally substituted by C₁-C₃ alkyl, —S(O)₂NH₂, —C(O)NR⁷R⁸ and CH₂OH,
provided that R¹ and R² are not both H and provided that when R² is other than H, R¹ is H; and when R¹ is other than H, R² is H.

3. A compound according to claim 1 or claim 2, wherein R⁴ is H or Me.

4. A compound according to any preceding claim, wherein R⁵ is H or F.

5. A compound according to any preceding claim, wherein R⁷ is H or Me.

6. A compound according to any preceding claim, wherein R¹³ is H.

7. A compound according to claim 1 which is selected from:
4-(3-[2,4′]Bipyridinyl-4-yl-imidazo[1,2-b]pyridazin-6-ylamino)-cyclohexanol;
4-{3-[2-(5-Methyl-thiophen-2-yl)-pyridin-4-yl]-imidazo[1,2-b]pyridazin-6-ylamino}-cyclohexanol;
4-[3-(2-Furan-3-yl-pyridin-4-yl)-imidazo[1,2-b]pyridazin-6-ylamino]-cyclohexanol;
4-{3-[2-(1-Methyl-1H-pyrazol-4-yl)-pyridin-4-yl]-imidazo[1,2-b]pyridazin-6-ylamino}-cyclohexanol;
4-[3-(4-Pyrazol-1-yl-phenyl)-imidazo[1,2-b]pyridazin-6-ylamino]-cyclohexanol;
4-[3-(2-Cyclopropyl-pyridin-4-yl)-imidazo[1,2-b]pyridazin-6-ylamino]-cyclohexanol;
4-[3-(3-Pyrazol-1-yl-phenyl)-imidazo[1,2-b]pyridazin-6-ylamino]-cyclohexanol;
4-[3-(4-[1,2,4]Triazol-1-yl-phenyl)-imidazo[1,2-b]pyridazin-6-ylamino]-cyclohexanol;
{4-[3-(4-Pyrazol-1-yl-phenyl)-imidazo[1,2-b]pyridazin-6-ylamino]-cyclohexyl}-methanol;
{4-[6-(2,5-Difluoro-benzylamino)-imidazo[1,2-b]pyridazin-3-yl]-phenyl}-(4-methyl-piperazin-1-yl)-methanone;
4-[6-(2,5-Difluoro-benzylamino)-imidazo[1,2-b]pyridazin-3-yl]-N-(2-morpholin-4-yl-ethyl)-benzamide;
{4-[6-(2,5-Difluoro-benzylamino)-imidazo[1,2-b]pyridazin-3-yl]-phenyl}-(4-dimethylamino-piperidin-1-yl)-methanone;
{4-[6-(2,5-Difluoro-benzylamino)-imidazo[1,2-b]pyridazin-3-yl]-phenyl}-morpholin-4-yl-methanone;
{3-[6-(2,5-Difluoro-benzylamino)-imidazo[1,2-b]pyridazin-3-yl]-N-(tetrahydro-pyran-4-yl)-benzamide;
4-[6-(2,5-Difluoro-benzylamino)-imidazo[1,2-b]pyridazin-3-yl]-N-(1-ethyl-pyrrolidin-2-ylmethyl)-benzamide;
4-[6-(2,5-Difluoro-benzylamino)-imidazo[1,2-b]pyridazin-3-yl]-N-(tetrahydro-pyran-4-yl)-benzamide;
4-[6-(3-Fluoro-benzylamino)-imidazo[1,2-b]pyridazin-3-yl]-benzenesulfonamide;
4-[6-(3-Fluoro-benzylamino)-imidazo[1,2-b]pyridazin-3-yl]-benzamide;
4-{6-[(R or S)-1-(3-Fluoro-phenyl)-2-hydroxy-ethylamino]-imidazo[1,2-b]pyridazin-3-yl}-benzonitrile;
3-{6-[(R)-1-(3-Fluoro-phenyl)-ethylamino]-imidazo[1,2-b]pyridazin-3-yl}-benzonitrile;
4-{6-[(R)-2-(3-Fluoro-phenyl)-pyrrolidin-1-yl]-imidazo[1,2-b]pyridazin-3-yl}-benzonitrile;
{4-[6-(3-Fluoro-benzyloxy)-imidazo[1,2-b]pyridazin-3-yl]-phenyl}-methanol; and
Tetrahydro-pyran-4-carboxylic acid {3-[6-(2,5-difluoro-benzylamino)-imidazo[1,2-b]pyridazin-3-yl]-phenyl}-amide.

8. A compound according to any one of claims 1 to 7 for use as a pharmaceutical.

9. A compound according to any one of claims 1 to 7 in combination with another drug substance which is an anti-inflammatory, a bronchodilator, an antihistamine, a decongestant or an anti-tussive drug substance.

10. A pharmaceutical composition comprising as active ingredient a compound according to any one of claims 1 to 7 and a suitable pharmaceutically acceptable excipient.

11. The use of a compound of Formula Ia or Ib according to any one of claims 1 to 7 for the manufacture of a medicament for the treatment of a condition mediated by one or more of ALK-5, Pi3K, TRK and JAK2.

12. The use of a compound of Formula Ia or Ib according to any one of claims 1 to 7 for the manufacture of a medicament for the treatment of a condition mediated by the ALK-4 receptor.

13. The use of a compound according to any one of claims 1 to 7 for the manufacture of a medicament for the treatment of pulmonary hypertension, chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, kidney fibrosis, lung fibrosis and liver fibrosis, hepatitis B, hepatitis C, alcohol-induced hepatitis, cancer, haemochromatosis, primary biliary cirrhosis, restenosis, retroperitoneal fibrosis, mesenteric fibrosis, endometriosis, keloids, cancer, abnormal bone function, inflammatory disorders, scarring and photaging of the skin.

14. The use of a compound according to any one of claims 1 to 7 for the manufacture of a medicament for the treatment of pulmonary hypertension or pulmonary fibrosis.

15. The use of a compound according to any one of claims 1 to 7 for the manufacture of a medicament for the treatment of osteoporosis.

16. A process for the preparation of a compound of Formula Ia or Ib as claimed in claim 1 which comprises:
(i) (A) reacting a compound of formula IIa 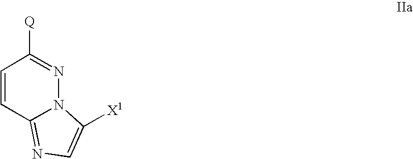
where Q is 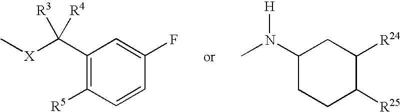
X, R³, R⁴, R⁵, R²⁴ and R²⁵ are as defined in claim 1,
and X¹ is halo, with a compound of formula IIIa or IIIb 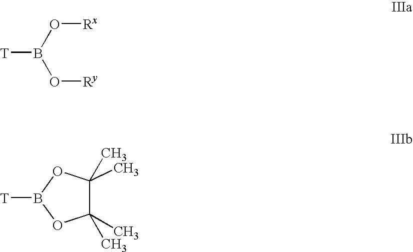
where T is 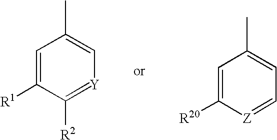
Y, Z, R¹, R² and R²⁰ are as defined in claim 1,
and R^{x} and R^{y} are independently hydrogen or C₁-C₈-alkyl;
(B) for the preparation of compounds of Formula Ia and Ib where Q includes a nitrogen linking group, reacting a compound of formula IV 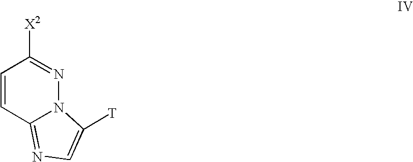
where T is as defined above and X² is halo, with a compound of formula V 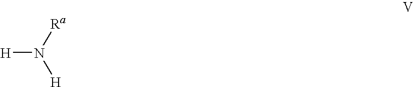
where R^{a} is 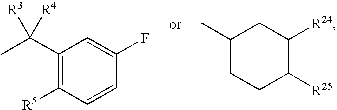
R³, R⁴, R⁵, R²⁴ and R²⁵ are as defined in claim 1;
(C) for the preparation of compounds of Formula Ia and Ib where Q includes a nitrogen or oxygen linking group and T is as defined above, reacting a compound of formula VI 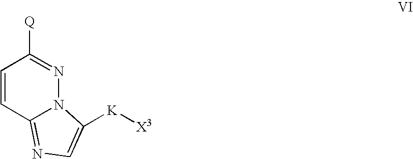
where Q is as defined above, K is a 6-membered heteroaromatic group and X³ is halo, with a compound of formula VIIa or VIIb 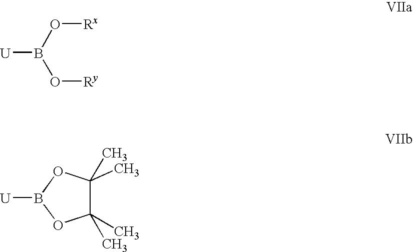
where U is —R¹, —R² or —R²⁰ and R^{x} and R^{y} are independently hydrogen or C₁-C₈-alkyl; or
(D) for the preparation of compounds of Formula Ia where Q includes an oxygen linking group, reacting a compound of formula IV where T is as defined above and X² is halo, with a compound of formula VIII
HO—R^{c} VIII
where R^{c} is a substituted benzyl group in accordance with the compounds as defined in claim 1; and
(ii) recovering the resultant compound of Formula Ia or Ib in free or salt or solvate form.
